(19) United States
(12) Patent Application Publication
(10) Pub. No.: US 2012/0322819 A1
(43) Pub. Date: Dec. 20, 2012

(54) COMPOUND CONTAINING A NOVEL 4-ALKOXYPYRIMIDINE STRUCTURE AND MEDICINE CONTAINING SAME

(75) Inventors: Toru Miura, Higashimurayama-shi (JP); Kazuhiro Onogi, Higashimurayama-shi (JP); Junya Tagashira, Higashimurayama-shi (JP); Gen Watanabe, Higashimurayama-shi (JP); Ryohei Sekimoto, Higashimurayama-shi (JP); Rie Ishida, Higashimurayama-shi (JP); Hitomi Aoki, Higashimurayama-shi (JP); Tadaaki Ohgiya, Higashimurayama-shi (JP)

(73) Assignee: KOWA COMPANY, LTD., Nagoya-shi, Aichi (JP)

(21) Appl. No.: 13/580,056

(22) PCT Filed: Feb. 25, 2011
(86) PCT No.: PCT/JP2011/001086

§ 371 Date: Aug. 20, 2012

(30) Foreign Application Priority Data
Feb. 25, 2010 (JP) 2010-040474

(51) Int. Cl. A61K 31/506; C07D 413/14; A61P 9/00; A61P 3/04; A61P 9/10; A61P 3/00; A61P 3/10; C07D 413/10; A61P 9/12

(52) U.S. Cl. 514/269; 544/319

(57) ABSTRACT

Disclosed is novel compound and medicinal formulation containing same, possessing both angiotensin II receptor blocking and PPARγ activation effect, of use as a medicine for prevention and/or treatment of hypertension, heart disease, angina pectoris, cerebrovascular disorder, cerebral circulatory disturbances, post-ischemic peripheral circulatory damage, renal disease, arteriosclerosis, inflammatory disorder, type 2 diabetes, diabetic complications, insulin resistance syndrome, syndrome X, metabolic syndrome and hyperinsulinaemia. Further disclosed is the general formula I (where one or both of R¹ and R² represent a C₁₋₆ alkyl group, R₃ represents a C₁₋₆ alkyl group which may contain one or more substituent groups selected from group A, or a C₃₋₈ cycloalkyl group which may contain one or more substituent groups selected from group B.) which represents the compound, and salts thereof, solvates thereof, and medicinal compositions containing any of these.

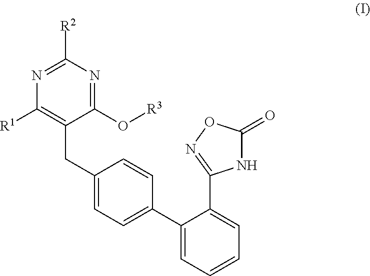

### TECHNICAL FIELD

The present invention relates to a novel compound having a structure of 4-alkoxypyrimidine that has both angiotensin II antagonistic activity and PPARγ activation activity, and a pharmaceutical agent containing the same.

### BACKGROUND ART

In recent years, diseases such as diabetes, hypertension, dyslipidemia and obesity which can be a risk factor for arteriosclerotic diseases have been rapidly increasing due to changes in life style with improvements in living standard, i.e., high calorie and high cholesterol type diet, obesity, lack of exercise, aging, and the like. It is known that, although being a risk factor independent of each other, overlap of the diseases can cause an occurrence of arteriosclerotic diseases at higher frequency or aggravation of the diseases. As such, with the understanding of a condition having a plurality of risk factors for arteriosclerotic diseases as metabolic syndrome, efforts have been made to elucidate the cause of the syndrome and to develop a therapeutic method therefor.

Angiotensin II (herein below, also abbreviated as “AII”) is a peptide that is found to be an intrinsic pressor substance produced by renin-angiotensin system (i.e., RA system). It is believed that pharmacological inhibition of angiotensin II activity can lead to treatment or prevention of circulatory diseases such as hypertension. Accordingly, an inhibitor for angiotensin converting enzyme (ACE) which inhibits the enzyme for promoting the conversion of angiotensin I (AI) to angiotensin II has been clinically used as an inhibitory agent for RA system. Furthermore, an orally administrable AII receptor blocker (Angiotensin Receptor Blocker: ARB) has been developed, and losartan, candesartan, telmisartan, valsartan, olmesartan, irbesartan, and the like are already clinically used as a hypotensive agent. It is reported by many clinical or basic studies that, as having not only a hypotensive activity but also other various activities including an anti-inflammatory activity, an endothelial function improving activity, a cardiovascular remodeling inhibiting activity, an oxidation stress inhibiting activity, a proliferation factor inhibiting activity, insulin resistance improving activity, and the like, ARB is useful for cardiovascular diseases, renal diseases, arteriosclerosis, and the like (Non-Patent Documents 1 and 2). Most recently, it is also reported that ARB particularly has a kidney protecting activity which does not depend on a hypotensive activity (Non-Patent Document 3).

Meanwhile, three isoforms, i.e., α, γ, and δ, have been identified so far for peroxisome proliferator-activated receptors (PPARγ) which belong to a nuclear receptor superfamily. Among them, PPARγ is an isoform most abundantly expressed in an adipose tissue and it plays an important role in differentiation of adipocytes or metabolism of glycolipids. Currently, thiazolidinedione derivatives (i.e., TZD) such as pioglitazone or rosiglitazone are clinically used as a therapeutic agent for diabetes having PPARγ activation activity, and they are known to have an activity of improving insulin resistance, glucose tolerance, lipid metabolism, and the like. Further, it is recently reported that, based on activation of PPARγ, TZD exhibits various activities including a hypotensive activity, an anti-inflammatory activity, an endothelial function improving activity, a proliferation factor inhibiting activity, an activity of interfering RA system, and the like. It is also reported that, according to such multiple activities, TZD shows a kidney protecting activity particularly in diabetic nephropathy without depending on blood sugar control (Non-Patent Documents 4, 5, 6, 7, and 8). Meanwhile, there is also a concern regarding adverse effects of TZD caused by PPARγ activation, such as body fluid accumulation, body weight gain, peripheral edema, and pulmonary edema (Non-Patent Documents 9 and 10).

It has been recently reported that telmisartan has a PPARγ activation activity (Non-Patent Document 11). It has been also reported that the irbesartan has the same activity (Non-Patent Document 12). These compounds have both an RA system inhibiting activity and a PPARγ activation activity, and thus are expected to be used as an integrated agent for prevention and/or treatment of circulatory diseases (e.g., hypertension, heart disease, angina pectoris, cerebral vascular accident, cerebrovascular disorder, ischemic peripheral circulatory disorder, kidney disease, and the like) or diabetes-related diseases (e.g., type 2 diabetes mellitus, diabetic complication, insulin resistant syndrome, metabolic syndrome, hyperinsulinemia, and the like) without increasing a risk of body fluid accumulation, body weight gain, peripheral edema, pulmonary edema, or congestive heart failure that are concerned over the use of TZD (Patent Document 1). Among them, for diabetic nephropathy, a synergistic prophylactic and/or therapeutic effect is expected from composited kidney protecting activity that is based on activities of RA system inhibition and PPARγ activation.

As a compound having the activities above, pyrimidine and triazine derivatives (Patent Document 1), imidazopyridine derivatives (Patent Document 2), indole derivatives (Patent Document 3), imidazole derivatives (Patent Document 4), and condensed ring derivatives (Patent Document 5) have been reported. However, there is no description or suggestion regarding a compound having 4-alkoxypyrimidine structure.

Meanwhile, an angiotensin II antagonist having 4-alkoxypyrimidine structure is known in literatures (Patent Documents 6 to 8 and Non-Patent Document 13). However, a compound having 1,2,4-oxadiazol-5(4H)-one structure, which is another characteristic of the invention, is not disclosed in any literatures, and also there is no description or suggestion relating to a PPARγ activation activity.

### PRIOR ART DOCUMENT

### Patent Document

Patent Document 1: WO 2008/062905

Patent Document 2: WO 2008/084303

Patent Document 3: WO 2008/096820

Patent Document 4: WO 2008/096829

Patent Document 5: WO 2008/143262

Patent Document 6: WO 1993/03018

Patent Document 7: Japanese Patent Application Laid-Open (JP-A) No. 4-230370

Patent Document 8: JP-A No. 3-133964

### Non-Patent Document

Non-Patent Document 1: AMER. J. Hypertension, 18, 720 (2005)

Non-Patent Document 2: Current Hypertension Report, 10, 261 (2008)

Non-Patent Document 3: Diabetes Care, 30, 1581 (2007)

Non-Patent Document 4: Kidney Int., 70, 1223 (2006)

Non-Patent Document 5: Circulation, 108, 2941 (2003)

Non-Patent Document 6:Best Pract. Res. Clin. Endocrinol. Metab., 21 (4), 687 (2007)

Non-Patent Document 7: Diab. Vasc. Dis. Res., 1 (2), 76 (2004)

Non-Patent Document 8: Diab. Vasc. Dis. Res., 2 (2), 61 (2005)

Non-Patent Document 9: J. Clin. Invest., 116 (3), 581 (2006)

Non-Patent Document 10: FASEB J., 20 (8), 1203 (2006)

Non-Patent Document 11: Hypertension, 43, 993 (2004)

Non-Patent Document 12: Circulation, 109, 2054 (2004)

Non-Patent Document 13: Eur. J. Med. Chem., 30, 365-375 (1995)

### SUMMARY OF THE INVENTION

### Problems to be Solved by the Invention

An object of the invention is to provide a novel compound that is useful as a pharmaceutical agent for preventing and/or treating hypertension as a circulatory disorder, diabetes as a metabolic disorder, and the like, and a pharmacological composition using the novel compound.

### Means for Solving the Problems

As a result of intensive studies to achieve the purpose described above, the inventors found that the compound represented by the formula (I) below has both an excellent angiotensin II antagonistic activity and an excellent PPARγ activation activity, and therefore completed the invention.

Specifically, the present invention relates to the following inventions.

[1]A compound represented by the formula (I) below or a salt thereof, or a solvate thereof:

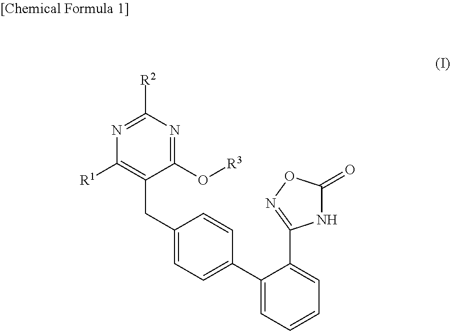

[in the formula, R¹ and R², which may be the same or different from each other, represent a C₁₋₆ alkyl group, and R³ represents a C₁₋₆ alkyl group which may have one or more substituents selected from the following Group A or a C₃₋₈ cycloalkyl group which may have one or more substituents selected from the following Group B],

Group A: a C₂₋₇ alkoxycarbonyl group; a C₁₋₆ alkoxy group which may have a substituent; a C₁₋₆ alkylthio group; a C₁₋₆ alkylsulfonyl group; a carboxy group; a carbamoyl group which may have one or more substituents; a hydroxy group; an oxo group; a dioxolanyl group; a pyrrolidinyl carbonyl group; a piperidinyl carbonyl group; a morpholinyl carbonyl group; an oxazolyl group which may have one or more substituents; and a C₆₋₁₀ aryl group which may have one or more substituents.

Group B: C₁₋₆ alkyl group; a hydroxy group; and an oxo group.

[2] The compound described in the above [1] or a salt thereof, or a solvate thereof, wherein the C₁₋₆ alkoxy group which may have a substituent is a C₁₋₆ alkoxy group, a C₁₋₆ alkoxy-C₁₋₆ alkoxy group, or a C₆₋₁₀ aryl-C₁₋₆ alkoxy group.

[3] The compound described in the above [1] or [2] or a salt thereof, or a solvate thereof, wherein the carbamoyl group which may have a substituent is a carbamoyl group or a C₁₋₆ alkyl-carbamoyl group.

[4] The compound described in the above [1] to [3] or a salt thereof, or a solvate thereof, wherein the oxazolyl group which may have a substituent is an oxazolyl group, a C₁₋₆ alkyl-oxazolyl group, or a C₆₋₁₀ aryl-oxazolyl group which may be substituted with a C₁₋₆ alkyl group.

[5] The compound described in the above [1] to [4] or a salt thereof, or a solvate thereof, wherein the C₆₋₁₀ aryl group which may have a substituent is a C₆₋₁₀ aryl group or a C₁₋₆ alkyl-C₆₋₁₀ aryl group which may be substituted with a C₁₋₆ alkyl group.

[6] The compound described in the above [1] to [5] or a salt thereof, or a solvate thereof, wherein the compound represented by the formula (I) is a compound selected from the group consisting of:

ethyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetate,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetic acid,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetamide,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylacetamide,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylacetamide, ethyl

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoate,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoic acid,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-butanamide,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylbutanamide,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylbutanamide,

3-{4′-{[4-butyl-6-(2-ethoxyethoxy)-2-methylpyrimidin-5-yl]methyl}-[1,1′-biphenyl]-2-yl}-1,2,4-oxadiazol-5(4H)-one,

3-{4′-{[4-butyl-2-methyl-6-(2-propoxyethoxy)pyrimidin-5-yl]methyl}-[1,1′-biphenyl]-2-yl}-1,2,4-oxadiazol-5(4H)-one,

3-{4′-{{4-butyl-6-[2-(2-methoxyethoxy)ethoxy]-2-methylpyrimidin-5-yl}methyl}-[1,1′-biphenyl]-2-yl}-1,2,4-oxadiazol-5 (4H)-one,

3-{4′-{{4-[2-(benzyloxy)ethoxy]-6-butyl-2-methylpyrimidin-5-yl}methyl}-[1,1′-biphenyl]-2-yl}-1,2,4-oxadiazol-5(4H)-one,

3-{4′-{[4-butyl-6-(2-hydroxyethoxy)-2-methylpyrimidin-5-yl]methyl}-[1,1′-biphenyl]-2-yl}-1,2,4-oxadiazol-5(4H)-one,

3-{4′-{[4-butyl-6-(2-isopropoxyethoxy)-2-methylpyrimidin-5-yl]methyl}-[1,1′-biphenyl]-2-yl}-1,2,4-oxadiazol-5(4H-one,

3-{4′-{{4-butyl-2-methyl-6-[2-(methylthio)ethoxy]pyrimidin-5-yl}methyl}-[1,1′-biphenyl]-2-yl}-1,2,4-oxadiazol-5(4H)-one,

3-{4′-{{4-butyl-2-methyl-6-[2-(methylsulfonyl)ethoxy]pyrimidin-5-yl}methyl}-[1,1′-biphenyl]-2-yl}-1,2,4-oxadiazo 1-5 (4H)-one,

3-{4′-{[4-butyl-6-(2,2-dimethoxyethoxy)-2-methylpyrimidin-5-yl]methyl}-[1,1′-biphenyl]-2-yl}-1,2,4-oxadiazol-5(4H)-one,

3-{4′-{[4-butyl-6-(2,2-diethoxyethoxy)-2-methylpyrimidin-5-yl]methyl}-[1,1′-biphenyl]-2-yl}-1,2,4-oxadiazol-5(4H)-one,

3-{4′-{[4-butyl-6-(2-hydroxypropoxy)-2-methylpyrimidin-5-yl]methyl}l-[1,1′-biphenyl]-2-yl}-1,2,4-oxadiazol-5(4H)-one,

3-{4′-{[4-butyl-2-methyl-6-(2-oxopropoxy)pyrimidin-5-yl]methyl}-[1,1′-biphenyl]-2-yl}-1,2,4-oxadiazol-5(4H)-one,

3-{4′-{[4-butyl-6-(4-methoxybutoxy)-2-methylpyrimidin-5-yl]methyl}-[1,1′-biphenyl]-2-yl}-1,2,4-oxadiazol-5(4H)-one,

3-{4′-{{4-[(1,3-dioxolan-2-yl)methoxy]-6-butyl-2-methylpyrimidin-5-yl}methyl}-[1,1′-biphenyl]-2-yl}-1,2,4-oxadiazol-5 (4H)-one,

methyl 2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetate,

2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetic acid,

methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionate,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionic acid,

3-{4′-{{4-butyl-2-methyl-6-[2-oxo-2-(pyrrolidin-1-yl)ethoxy]pyrimidin-5-yl}methyl}-[1,1′-biphenyl]-2-yl}-1,2,4-oxadiazol-5 (4H)-one,

3-{4′-{{4-butyl-2-methyl-6-[2-oxo-2-(piperidin-1-yl)ethoxy]pyrimidin-5-yl}methyl}-[1,1′-biphenyl]-2-yl}-1,2,4-oxadiazol-5 (4H)-one,

3-{4′-{[4-butyl-2-methyl-6-(2-morpholino-2-oxoethoxy)pyrimidin-5-yl]methyl}-[1,1′-biphenyl]-2-yl}-1,2,4-oxadiazo 1-5 (4H)-one,

3-{4′-{{4-butyl-2-methyl-6-{[5-methyl-2-(p-tolyl)oxazol-4-yl]methoxy}pyrimidin-5-yl}methyl}-[1,1′-biphenyl]-2-yl}-1,2,4-oxadiazol-5(4H)-one,

3-{4′-{[4-butyl-6-(2-methoxyethoxy)-2-methylpyrimidin-5-yl]methyl}-[1,1′-biphenyl]-2-yl}-1,2,4-oxadiazol-5(4H)-one,

3-{4′-{{4-butyl-6-[(4-hydroxycyclohexyl)oxy]-2-methyl pyrimidin-5-yl}methyl}-[1,1′-biphenyl]-2-yl}-1,2,4-oxadiazo 1-5 (4H)-one,

3-{4′-{{4-butyl-2-methyl-6-[(4-oxocyclohexyl)oxy]pyrimidin-5-yl}methyl}-[1,1′-biphenyl]-2-yl}-1,2,4-oxadiazol-5(4H)-one,

3-{4′-{{4-butyl-6-[3-methoxy-4-(2-methoxyethoxy)phenethoxy]-2-methylpyrimidin-5-yl}methyl}-[1,1′-biphenyl]-2-yl}-1,2,4-oxadiazol-5(4H)-one, and

3-{4′-{[4-(2-methoxyethoxy)-2-methyl-6-pentylpyrimidin-5-yl]methyl}-[1,1′-biphenyl]-2-yl}-1,2,4-oxadiazol-5(4H)-one.

The alkyl group such as butyl in the nomenclature of the above-mentioned compounds represents a straight (normal) unless particularly described.

[7] A pharmaceutical composition containing the compound or the salt thereof, or the solvate thereof described in the above [1] to [6], and a pharmaceutically acceptable carrier.

[8] A pharmaceutical composition containing the compound or the salt thereof, or the solvate thereof described in the above [1] to [6] as an effective component, having both angiotensin II receptor antagonistic activity and PPARγ activation activity.

[9] An agent for preventing and/or treating a circulatory disease containing as an effective component the compound or the salt thereof, or the solvate thereof described in the above [1] to [6].

[10] The agent for preventing and/or treating a circulatory disease described in the above [9], wherein the circulatory disease is hypertension, heart disease, angina pectoris, cerebral vascular accident, cerebrovascular disorder, ischemic peripheral circulatory disorder, kidney disease, or arteriosclerosis.

[11] An agent for preventing and/or treating a metabolic disease containing as an effective component the compound or the salt thereof, or the solvate thereof described in the above [1] to [6].

[12] The agent for preventing and/or treating a metabolic disease described in the above [11], wherein the metabolic disease is type 2 diabetes mellitus, diabetic complication (diabetic retinopathy, diabetic neuropathy, diabetic nephropathy), insulin resistant syndrome, metabolic syndrome, or hyperinsulinemia.

[13] A method of preventing and/or treating a circulatory disease characterized in that an effective amount of the compound or the salt thereof, or the solvate thereof described in the above [1] to [6] is administered to a patient in need of the treatment.

[14] The method of preventing and/or treating a circulatory disease described in the above [13], wherein the circulatory disease is hypertension, heart disease, angina pectoris, cerebral vascular accident, cerebrovascular disorder, ischemic peripheral circulatory disorder, kidney disease, or arteriosclerosis.

[15] A method of preventing and/or treating a metabolic disease characterized in that an effective amount of the compound or the salt thereof, or the solvate thereof described in the above [1] to [6] is administered to a patient in need of the treatment.

[16] The method of preventing and/or treating a metabolic disease described in the above [15], wherein the metabolic disease is type 2 diabetes mellitus, diabetic complication (diabetic retinopathy, diabetic neuropathy, diabetic nephropathy), insulin resistant syndrome, metabolic syndrome, or hyperinsulinemia.

[17] Use of the compound or the salt thereof, or the solvate thereof described in the above [1] to [6] for production of a preparation used for prevention and/or treatment of a circulatory disease.

[18] The use of the compound or the salt thereof, or the solvate thereof described in the above [17], wherein the circulatory disease is hypertension, heart disease, angina pectoris, cerebral vascular accident, cerebrovascular disorder, ischemic peripheral circulatory disorders, kidney disease, or arteriosclerosis.

[19] Use of the compound or the salt thereof, or the solvate thereof described in the above [1] to [6] for production of a preparation used for prevention and/or treatment of a metabolic disease.

[20] The use of the compound or the salt thereof, or the solvate thereof described in the above [19], wherein the metabolic disease is type 2 diabetes mellitus, diabetic complication (diabetic retinopathy, diabetic neuropathy, diabetic nephropathy), insulin resistant syndrome, metabolic syndrome, or hyperinsulinemia.

[21] The compound or the salt thereof, or the solvate thereof described in the above [1] to [6] as a preventive and/or therapeutic agent having both an angiotensin II receptor antagonistic activity and PPARγ activation activity.

### Effects of the Invention

The 4-Alkoxypyrimidine derivative represented by the formula (I) of the invention or a salt thereof, or a solvate thereof exhibits a potent antagonistic activity for an angiotensin II receptor, and can be appropriately used as an effective component for an agent for preventing and/or treating a disease related with angiotensin II, for example a circulatory disease such as hypertension, heart disease, angina pectoris, cerebral vascular accident, cerebrovascular disorder, ischemic peripheral circulatory disorder, kidney disease, and arteriosclerosis.

Further, the 4-alkoxypyrimidine derivative represented by the formula (I) of the invention or a salt thereof, or a solvate thereof has a PPARγ activation activity and can be appropriately used as an effective component for an agent for preventing and/or treating a disease related with PPARγ, for example a metabolic disease such as arteriosclerosis, type 2 diabetes mellitus, diabetic complication (diabetic retinopathy, diabetic neuropathy, or diabetic nephropathy), insulin resistance syndrome, syndrome X, metabolic syndrome, and hyperinsulinemia.

Still further, the 4-alkoxypyrimidine derivative represented by the formula (I) of the invention, or a salt thereof, or a solvate thereof has both an antagonistic activity for an angiotensin II receptor and a PPARγ activation activity and can be appropriately used as an effective component for an agent for preventing and/or treating a disease related with both angiotensin II and PPARγ, for example, arteriosclerosis, diabetic nephropathy, insulin resistance syndrome, syndrome X, and metabolic syndrome.

### Modes for Carrying out the Invention

The “halogen atom” as used herein includes a fluorine atom, a chlorine atom, a bromine atom, an iodine atom, and the like.

The “C₁₋₆ alkyl group” as used herein means a linear or a branched saturated hydrocarbon group having 1 to 6 carbon atoms, and examples thereof include a methyl group, an ethyl group, a propyl group, an isopropyl group, a n-butyl group, an isobutyl group, a sec-butyl group, a tert-butyl group, a pentyl group, an isopentyl group, a neopentyl group, a hexyl group, an isohexyl group, and the like.

The “C₃₋₈ cycloalkyl group” as used herein means a saturated cycloalkyl group having 3 to 8 carbon atoms, and examples thereof include a cyclopropyl group, a cyclobutyl group, a cyclopentyl group, a cyclohexyl group, a cyclopentyl group, a cyclooctyl group, and the like.

The “C₁₋₆ alkoxy group” as used herein means a linear or a branched alkoxy group having 1 to 6 carbon atoms, and examples thereof include a methoxy group, an ethoxy group, a propoxy group, an isopropoxy group, a butoxy group, an isobutoxy group, a sec-butoxy group, a tert-butoxy group, a pentoxy group, an isopentoxy group, a neopentoxy group, a hexyloxy group, an isohexyloxy group, and the like.

The “C₂₋₇ alkoxycarbonyl group” as used herein means a carbonyl group to which the “C₁₋₆alkoxy group” described above is bonded, and examples thereof include a methoxycarbonyl group, an ethoxycarbonyl group, a propoxycarbonyl group, an isopropoxycarbonyl group, a butoxycarbonyl group, an isobutoxycarbonyl group, a sec-butoxy group, a tert-butoxycarbonyl group, a pentoxycarbonyl group, an isopentoxycarbonyl group, a neopentoxycarbonyl group, a hexyloxycarbonyl group, an isohexyloxycarbonyl group, and the like. Further, number of the carbon atoms described herein means the number of carbon atoms including the carbon atom in a carbonyl group.

The “C₁₋₆ alkylthio group” as used herein means a linear or a branched alkylthio group having 1 to 6 carbon atoms, and examples thereof include a methylthio group, an ethylthio group, a propylthio group, a butylthio group, a pentylthio group, a hexylthio group, and the like.

The “C₂₋₆ alkylsulfonyl group” as used herein means a sulfonyl group (SO₂) to which the “C₂₋₆ alkyl group” described above is bonded, and examples thereof include a methylsulfonyl group, an ethylsulfonyl group, a propylsulfonyl group, a butylsulfonyl group, a pentylsulfonyl group, a hexylsulfonyl group, and the like.

When used herein, examples of the “C₆₋₁₀ aryl group” include a phenyl group, a naphthyl group, an azulenyl group, and the like.

Examples of the preferred mode of the invention include the followings.

In the formula (I), the C₁₋₆ alkyl group in R¹ is preferably a C₃₋₆ alkyl group, and more preferably a butyl group.

In the formula (I), the C₁₋₆ alkyl group in R² is preferably a C₁₋₄ alkyl group, and more preferably a methyl group.

In the formula (I), the C₁₋₆ alkyl group in R³ is preferably a C₁₋₄ alkyl group.

In the formula (I), the C₃₋₈ cycloalkyl group in R³ is preferably a C₃₋₆ cycloalkyl group, and more preferably a cyclohexyl group.

In the formula (I), the C₂₋₇ alkoxycarbonyl group is preferably a C₂₋₄ alkoxycarbonyl group, and more preferably an ethoxycarbonyl group.

In the formula (I), examples of the substituent for the C₁₋₆ alkoxy group which may have a substituent include a substituent selected from a group (herein below, referred to as “Group A”) consisting of a C₂₋₇ alkoxycarbonyl group; a C₁₋₆ alkoxy group which may have a substituent; a C₁₋₆ alkylthio group; a C₁₋₆ alkylsulfonyl group; a carboxy group; a carbamoyl group which may have one or more substituents; a hydroxy group; an oxo group; a dioxolanyl group; a pyrrolidinylcarbonyl group; a piperidinylcarbonyl group; a morpholinylcarbonyl group; an oxazolyl group which may have one or more substituents; and a C₆₋₁₀ aryl group which may have one or more substituents. Preferred examples thereof include a C₁₋₆ alkoxy group which may have a substituent and a C₆₋₁₀ aryl group which may have a substituent. Preferred examples of the C₁₋₆ alkoxy group which may have a substituent include a C₁₋₆ alkoxy group, a C₁₋₆ alkoxy-C₁₋₆ alkoxy group, and a C₆₋₁₀ aryl-C₁₋₆ alkoxy group. Preferred examples of the C₁₋₆ alkoxy group include a C₁₋₄ alkoxy group. More preferred examples thereof include a methoxy group, an ethoxy group, a propoxy group, and an isopropoxy group. Preferred examples of the C₁₋₆ alkoxy-C₁₋₆ alkoxy group include a C₁₋₄ alkoxy-C₁₋₄ alkoxy group. More preferred examples thereof include a methoxyethoxy group. Preferred examples of the C₆₋₁₀ aryl-C₁₋₆ alkoxy group include a C₆₋₁₀ aryl-C₁₋₄ alkoxy group. More preferred examples thereof include a benzyloxy group.

Preferred examples of the C₁₋₆ alkylthio group in Group A above include a C₁₋₄ alkylthio group. More preferred examples thereof include a methylthio group.

Preferred examples of the C₁₋₆ alkylsulfonyl group in Group A above include a C₁₋₄ alkylsulfonyl group. More preferred examples thereof include a methylsulfonyl group.

Preferred examples of the substituent for the carbamoyl group which may have one or more substituents in Group A include a C₁₋₆ alkyl group. Preferred examples of the carbamoyl group which may have one or more substituents include a carbamoyl group, a mono(C₁₋₆ alkyl)carbamoyl group, and a di(C₁₋₆ alkyl)carbamoyl group. Preferred examples of the mono(C₁₋₆ alkyl)carbamoyl group include a mono(C₁₋₄ alkyl)carbamoyl group. More preferred examples thereof include an ethylcarbamoyl group. Preferred examples of the di(C₁₋₆ alkyl)carbamoyl group include a di(C₁₋₄ alkyl)carbamoyl group. More preferred examples thereof include a diethylcarbamoyl group.

Preferred examples of the substituents for the oxazolyl group which may have one or more substituents in Group A above include a C₁₋₆ alkyl group and a C₆₋₁₀ aryl group which may be substituted with a C₁₋₆ alkyl group. Preferred examples of the C₁₋₆ alkyl group include C₁₋₄ alkyl group. More preferred examples thereof include a methyl group. Preferred examples of the C₆₋₁₀ aryl group which may be substituted with a C₁₋₆ alkyl group include a C₁₋₄ alkyl C₆₋₁₀ aryl group. More preferred examples thereof include a C₁₋₄ alkyl-phenyl group such as a methylphenyl group.

Preferred examples of the substituent for the C₆₋₁₀ aryl group which may have one or more substituents in Group A above include a C₁₋₆ alkoxy group and a C₁₋₆ alkoxy C₁₋₆ alkoxy group. Preferred examples of the C₁₋₆ alkoxy group include a C₁₋₄ alkoxy group. More preferred examples thereof include a methoxy group. Preferred examples of the C₁₋₆ alkoxy C₁₋₆ alkoxy group include a C₁₋₄ alkoxy C₁₋₄ alkoxy group. More preferred examples thereof include a methoxyethoxy group.

In the formula (I), examples of the substituent for the C₃₋₈ cycloalkyl group which may have a substituent include a group selected from a group consisting of C₁₋₆ alkyl group; a hydroxy group; and an oxo group (herein below, referred to as “Group B”).

More preferred examples of the compound represented by the formula (I) include a compound selected from a group consisting of the following compounds:

ethyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetate,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetic acid,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetamide,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethyl acetamide,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylacetamide,

ethyl 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoate,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoic acid,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-butanamide,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylbutanamide,

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylbutanamide,

3-{4′-{[4-butyl-6-(2-ethoxyethoxy)-2-methylpyrimidin-5-yl]methyl}-[1,1′-biphenyl]-2-yl}-1,2,4-oxadiazol-5(4H)-one,

3-{4′-{[4-butyl-2-methyl-6-(2-propoxyethoxy)pyrimidin-5-yl]methyl}-[1,1′-biphenyl]-2-yl}-1,2,4-oxadiazol-5(4H)-one,

3-{4′-{{4-butyl-6-[2-(2-methoxyethoxy)ethoxy]-2-methylpyrimidin-5-yl}methyl}-[1,1′-biphenyl]-2-yl}-1,2,4-oxadiazol-5 (4H)-one,

3-{4′-{{4-[2-(benzyloxy)ethoxy]-6-butyl-2-methylpyrimidin-5-yl}methyl}-[1,1′-biphenyl]-2-yl}-1,2,4-oxadiazol-5(4H)-one,

3-{4′-{[4-butyl-6-(2-hydroxyethoxy)-2-methylpyrimidin-5-yl]methyl}-[1,1′-biphenyl]-2-yl}-1,2,4-oxadiazol-5(4H)-one,

3-{4′-{[4-butyl-6-(2-isopropoxyethoxy)-2-methylpyrimidin-5-yl]methyl}-[1,1′-biphenyl]-2-yl}-1,2,4-oxadiazol-5(4H-one,

3-{4′-{{4-butyl-2-methyl-6-[2-(methylthio)ethoxy]pyrimidin-5-yl}methyl}-[1,1′-biphenyl]-2-yl}-1,2,4-oxadiazol-5(4H)-one,

3-{4′-{{4-butyl-2-methyl-6-[2-(methylsulfonyl)ethoxy]pyrimidin-5-yl}methyl}-[1,1′-biphenyl]-2-yl}-1,2,4-oxadiazol-5 (4H)-one,

3-{4′-{[4-butyl-6-(2,2-dimethoxyethoxy)-2-methylpyrimidin-5-yl]methyl}-[1,1′-biphenyl]-2-yl}-1,2,4-oxadiazol-5(4H)-one,

3-{4′-{[4-butyl-6-(2,2-diethoxyethoxy)-2-methylpyrimidin-5-yl]methyl}l-[1,1′-biphenyl]-2-yl}-1,2,4-oxadiazol-5(4H-one,

3-{4′-{[4-butyl-6-(2-hydroxypropoxy)-2-methylpyrimidin-5-yl]methyl}-[1,1′-biphenyl]-2-yl}-1,2,4-oxadiazol-5(4H)-one,

3-{4′-{[4-butyl-2-methyl-6-(2-oxopropoxy)pyrimidin-5-yl]methyl}-[1,1′-biphenyl]-2-yl}-1,2,4-oxadiazol-5(4H)-one,

3-{4′-{[4-butyl-6-(4-methoxybutoxy)-2-methylpyrimidin-5-yl]methyl}-[1,1′-biphenyl]-2-yl}-1,2,4-oxadiazol-5(4H)-one,

3-{4′-{{4-[(1,3-dioxolan-2-yl)methoxy]-6-butyl-2-methylpyrimidin-5-yl}methyl}-[1,1′-biphenyl]-2-yl}-1,2,4-oxadiazol-5(4H)-one,

methyl 2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetate,

2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetic acid,

methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionate,

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionic acid,

3-{4′-{{4-butyl-2-methyl-6-[2-oxo-2-(pyrrolidin-1-yl)ethoxy]pyrimidin-5-yl}methyl}-[1,1′-biphenyl]-2-yl}-1,2,4-oxadiazol-5(4H)-one,

3-{4′-{{4-butyl-2-methyl-6-[2-oxo-2-(piperidin-1-yl)ethoxy]pyrimidin-5-yl}methyl}-[1,1′-biphenyl]-2-yl}-1,2,4-oxadiazol-5 (4H)-one,

3-{4′-{[4-butyl-2-methyl-6-(2-morpholino-2-oxoethoxy)pyrimidin-5-yl]methyl}-[1,1′-biphenyl]-2-yl}-1,2,4-oxadiazol-5 (4H)-one,

3-{4′-{{4-butyl-2-methyl-6-{[5-methyl-2-(p-tolyl)oxazof-4-yl]methoxy}pyrimidin-5-yl}methyl}-[1,1′-biphenyl]-2-yl}-1,2,4-oxadiazol-5(4H)-one,

3-{4′-{[4-butyl-6-(2-methoxyethoxy)-2-methylpyrimidin-5-yl]methyl}-[1,1′-biphenyl]-2-yl}-1,2,4-oxadiazol-5(4H)-one,

3-{4′-{{4-butyl-6-[(4-hydroxycyclohexyl)oxy]-2-methyl pyrimidin-5-yl]methyl}-[1,1′-biphenyl]-2-yl}-1,2,4-oxadiazol-5 (4H)-one,

3-{4′-{{4-butyl-2-methyl-6-[(4-oxocyclohexyl)oxy]pyrimidin-5-yl}methyl}-[1,1′-biphenyl]-2-yl}-1,2,4-oxadiazol-5(4H)-one,

3-{4′-{{4-butyl-6-[3-methoxy-4-(2-methoxyethoxy)phenethoxy]-2-methylpyrimidin-5-yl}methyl}-[1,1′-biphenyl]-2-yl}-1,2,4-oxadiazol-5(4H)-one, and

3-{4′-{[4-(2-methoxyethoxy)-2-methyl-6-pentylpyrimidin-5-yl]methyl}-[1,1′-biphenyl]-2-yl}-1,2,4-oxadiazol-5(4H)-one.

If the compound of the invention has geometrical isomers or optical isomers, the invention encompasses all of such isomers. Isolation of these isomers is carried out by an ordinary method.

Salts of the compound represented by the formula (I) are not particularly limited, if they are pharmaceutically acceptable salts. When the compound is processed as an acidic compound, an alkali metal salt or an alkali earth metal salt such as sodium salt, potassium salt, magnesium salt, and calcium salt, and the like; and a salt with an organic base such as trimethylamine, triethylamine, pyridine, picoline, N-methylpyrrolidine, N-methylpiperidine, N-methylmorpholine, and the like can be mentioned. When the compound is processed as a basic compound, an acid addition salt and the like including a salt with a mineral acid, for example, hydrochloric acid salt, hydrobromic acid salt, hydroiodic acid salt, sulfuric acid salt, nitric acid salt, phosphoric acid salt, and the like; or organic acid addition salt, for example, benzoic acid salt, methanesulfonic acid salt, ethanesulfonic acid salt, benzenesulfonic acid salt, p-toluene sulfonic acid salt, maleic acid salt, fumaric acid salt, tartaric acid salt, citric acid salt, and acetic acid salt; or the like can be mentioned.

Examples of the solvate of the compound represented by the formula (I) or a salt thereof include a hydrate, but not limited thereto.

In addition, compounds which are metabolized in a living body and converted into the compounds represented by the aforementioned formula (I), so called prodrugs, all fall within the scope of the compounds of the invention. Examples of groups which form the prodrugs of the compounds of the invention include the groups described in “Progress in Medicine”, Vol. 5, pp. 2157-2161, 1985, Life Science Medica, and the groups described in “Development of Drugs”, Vol. 7, Molecular Designs, pp. 163-198, 1990, Hirokawa Shoten.

The compounds represented by the formula (I), or salts or solvates thereof can be produced according to various known methods, and the production method is not specifically limited. For example, the compounds can be produced according to the following reaction step. Further, when each reaction shown below is performed, functional groups other than the reaction sites may be protected beforehand as required, and deprotected in an appropriate stage. Furthermore, the reaction in each step may be performed by an ordinarily used method, and isolation and purification can be performed by a method suitably selected from conventional methods such as crystallization, recrystallization, chromatography, or the like, or a combination thereof.

### (Production Method)

Among the compounds that are represented by the formula (I) of the invention, the compound represented by the formula (Ia) can be produced by the method described below, for example, but not limited thereto. Specifically, as described in the Reaction Scheme 1 below, the pyrimidinone derivative (II) is reacted with the alkyl halide (III) or the alcohol (IV), and subsequently the resulting compound (V) is reacted with hydroxylamine to give the amide oxime (VI). By further reacting the amide oxime (VI) with a carbonyl reagent, the compound represented by the formula (Ia) of the invention can be produced.

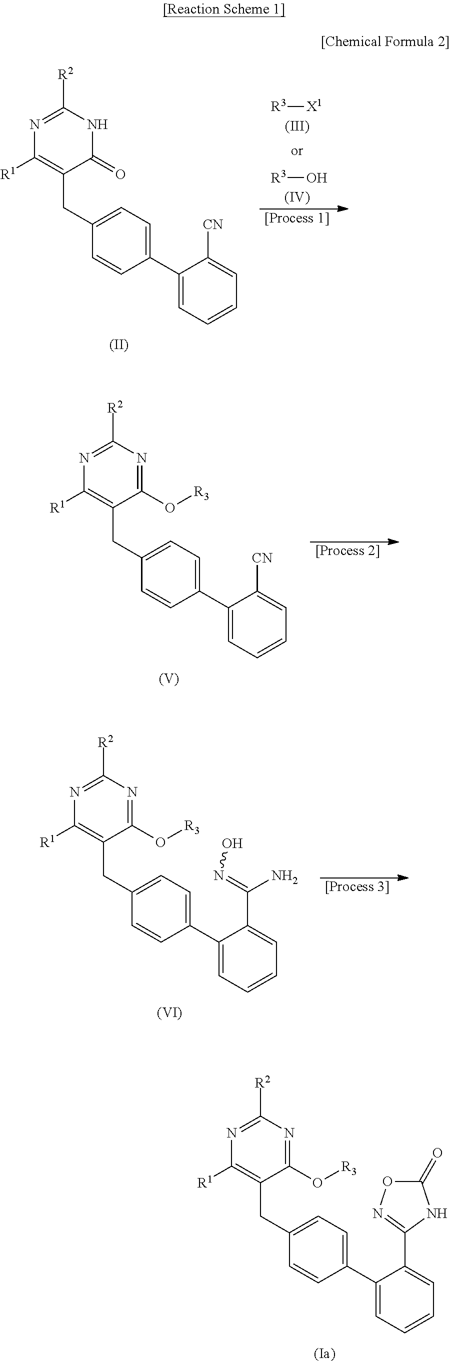

[in the formula, R², R², and R³ are as defined above and X² represents a halogen atom]

### [Process 1]

The reaction between the pyrimidinone derivative (II) and the halide (III) maybe carried out in a solvent in the presence or absence of a base. The solvent is not specifically limited, and tetrahydrofuran, toluene, dioxane, N,N-dimethylformamide, N-methylpyrrolidone, dichloromethane, chloroform, acetonitrile, propionitrile, and the like may be used either alone or in combination thereof. The base is not specifically limited, and examples thereof include an organic base such as pyridine, N,N-dimethylaminopyridine (DMAP), collidine, lutidine, 1,8-diazabicyclo[5.4.0]-7-undecene (DBU), 1,5-diazabicyclo[4.3.0]-5-nonene (DBN), 1,4-diazabicyclo[2.2.2]octane (DABCO), triethylamine, diisopropylamine, diisopropylethylamine, diisopropylpentylamine, trimethylamine, and the like, an alkali metal hydride such as a lithium hydride, sodium hydride, potassium hydride, and the like, an alkali metal hydroxide such as lithium hydroxide, sodium hydroxide, potassium hydroxide, and the like, an alkali metal carbonate such as lithium carbonate, sodium carbonate, potassium carbonate, cesium carbonate, and the like, and sodium hydrogen carbonate. The reaction condition may vary depending on the reaction materials used. However, the reaction is generally carried out at −20° C. to 100° C., preferably 15° C. to 80° C. for 5 minutes to 36 hours, and preferably for 5 hours to 24 hours to obtain the compound (V).

The compound represented by the formula (V) can be also produced based on Mitsunobu reaction using the alcohol (IV). The reaction between the compound (II) and the alcohol compound (IV) can be carried out in a solvent by using a phosphine reagent and an azo reagent or an ethylene dicarboxylic acid reagent or a phosphonium ylide reagent. The phosphine reagent is not specifically limited, and trialkylphosphine or triarylphosphines, particularly trimethylphosphine, triethylphosphine, tripropylphosphine, triisopropylphosphine, tributylphosphine, triisobutylphosphine, tricyclohexylphosphine, triphenylphosphine, diphenylphosphino polystyrene, and the like may be used. The azo reagent is not specifically limited, and diethyl azodicarboxylate (DEAD), diisopropyl azodicarboxylate, 1,1-(azodicarbonyl)piperidine (ADPP), 1,1′-azobis(N,N′-diisopropyl formamide) (TIPA), 1,6-dimethyl-1,5,7-hexahydro-1,4,6-tetrazocine-2,5-dione (DHAD), and the like may be used. The ethylene dicarboxylic acid reagent is not specifically limited, and dimethyl maleate, diethyl maleate, dimethyl fumarate, diethyl fumarate, and the like maybe used. The solvent is not specifically limited, and N,N′-dimethylformamide, tetrahydrofuran, dioxane, acetonitrile, propionitrile, nitromethane, acetone, ethyl acetate, isopropyl acetate, benzene, toluene, chlorobenzene, chloroform, dichloromethane, 1,2-dichloroethane, and the like may be used either alone or in combination thereof. The reaction condition may vary depending on the reaction materials used. However, the reaction is generally carried out at 0° C. to 120° C., preferably 0° C. to 100° C. for 30 minutes to 3 days, and preferably 30 minutes to 50 hours to obtain the compound (V).

### [Process 2]

The reaction between the compound (V) and hydroxylamine can be carried out in a solvent. The solvent is not specifically limited, and N,N-dimethylformamide, N,N-dimethylacetamide, N-methylpyrrolidone, dimethylsulfoxide, methanol, ethanol, isopropanol, 1,4-dioxane, tetrahydrofuran, or the like may be used either alone or in combination thereof. When an acid salt such as hydroxylamine hydrochloride, hydroxylamine sulfuric acid, hydroxylamine oxalic acid, and the like is used as hydroxylamine, a suitable base, for example, potassium carbonate, sodium hydrogen carbonate, sodium hydroxide, triethylamine, sodium methoxide, sodium hydride, and the like maybe used in an equivalent amount or a slightly excess amount for the reaction. The reaction condition may vary depending on the reaction materials used. However, the reaction is generally carried out at 0° C. to 180° C., preferably 50° C. to 120° C., for 1 minute to 3 days, and preferably for 1 hour to 36 hours to obtain the amide oxime (VI).

### [Process 3]

Conversion of the amide oxime (VI) to the compound (Ia) can be carried out in a solvent in the presence of a base by using a carbonylation reagent. The solvent is not specifically limited, and 1,2-dichloroethane, chloroform, dichloromethane, ethyl acetate, isopropyl acetate, toluene, benzene, tetrahydrofuran, dioxane, acetonitrile, propionitrile, N,N-dimethylformamide, N,N-dimethylacetamide, N-methylpyrrolidone, diethyl ether, or the like may be used either alone or in combination thereof. The base is not specifically limited, and examples thereof include pyridine, DMAP, collidine, lutidine, DBU, DBN, DABCO, triethylamine, diisopropylethylamine, diisopropylpentylamine, trimethylamine, lithium carbonate, sodium carbonate, potassium carbonate, cesium carbonate, sodium hydrogen carbonate, potassium hydrogen carbonate, or the like. The carbonylation reagent is not specifically limited, and 1,1′-carbonyldiimidazole, triphosgene, methyl chlorocarbonate, ethyl chlorocarbonate, or the like maybe used. The reaction condition may vary depending on the reaction materials used. However, the reaction is generally carried out at 0° C. to 120° C., preferably 15° C. to 80° C. for 5 minutes to 3 days, and preferably for 30 minutes to 12 hours to obtain the compound (Ia).

Further, among the compounds represented by the formula (V), the compound represented by the formula (VIII) or the formula (X) can be produced according to the method described below, but not limited thereto.

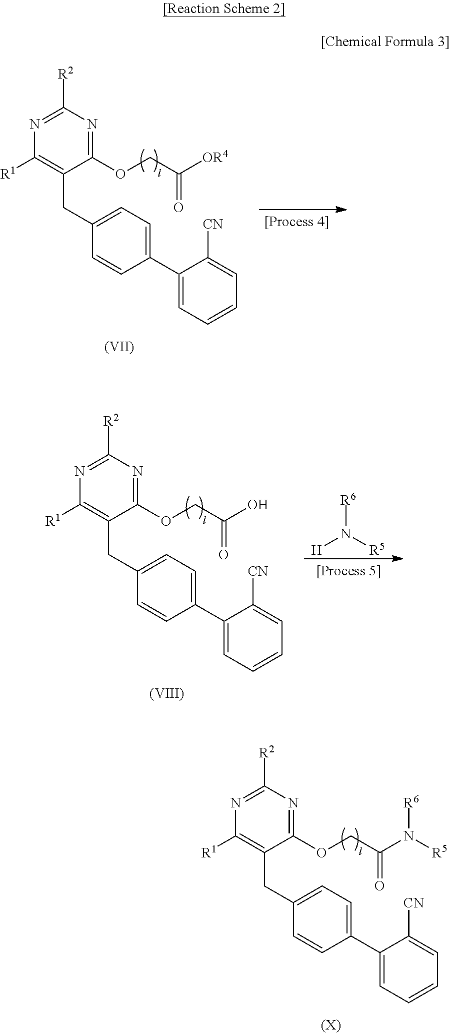

[in the formula, R¹ and R² are as defined above, R⁴ represents a C₁₋₆ alkyl group, R⁵ and R⁶ represent a hydrogen atom or a C₁₋₆ alkyl group, and 1 represents an integer of from 1 to 6].

### [Process 4]

The carboxylic acid compound (VIII) can be obtained by carrying out common hydrolysis of the carboxylic acid derivative (VII). The reaction can be carried out in a solvent in the presence of a base or an acid. The solvent is not specifically limited, and tetrahydrofuran, dioxane, methanol, ethanol, water, or the like may be used either alone or in combination thereof. The base is not specifically limited, and examples thereof include an alkali metal hydroxide such as lithium hydroxide, sodium hydroxide, potassium hydroxide, and the like, an alkali metal carbonate such as lithium carbonate, and sodium carbonate, potassium carbonate, cesium carbonate, and the like, and potassium trimethylsilanolate. The acid is not specifically limited, and hydrochloric acid, acetic acid, trifluoroacetic acid, boron tribromide, aluminum chloride, and the like maybe used. The reaction condition may vary depending on the reaction materials used. However, the reaction is generally carried out at −20° C. to 100° C., preferably 15° C. to 80° C. for 5 minutes to 1 day, and preferably for 30 minutes to 13 hours to obtain the carboxylic acid compound (VIII).

### [Process 5]

The dehydration condensation reaction between the carboxylic acid compound (VIII) and the amine compound (IX) can be carried out by using a condensation agent in a solvent in the presence or absence of a base and/or a condensation promoting agent. The solvent is not specifically limited, and examples thereof include 1,2-dichloroethane, chloroform, dichloromethane, ethyl acetate, isopropyl acetate, toluene, benzene, tetrahydrofuran, dioxane, acetonitrile, propionitrile, N,N-dimethylformamide, N-methylpyrrolidone, or the like. The base is not specifically limited, and examples thereof include pyridine, DMAP, collidine, lutidine, DBU, DBN, DABCO, triethylamine, diisopropylethylamine, diisopropylpentylamine, trimethylamine, lithium hydride, sodium hydride, potassium hydride, lithium hydroxide, sodium hydroxide, potassium hydroxide, lithium carbonate, sodium carbonate, potassium carbonate, cesium carbonate, sodium hydrogen carbonate, potassium hydrogen carbonate, and the like. The condensation promoting agent is not specifically limited, and examples thereof include DMAP, 1-hydroxy-7-azabenzotriazole (HOAt), 1-hydroxybenzotriazole (HOBt), 3-hydroxy-3,4-dihydro-4-oxo-1,2,3-benzotriazole (HODhbt), N-hydroxy-5-norbornene-2,3-dicarboximide (HONB), pentafluorophenol (HOPfp), N-hydroxyphthalimide (HOPht), N-hydroxysuccinimide (HOSu), or the like. The condensation agent is not specifically limited, and examples thereof include N,N′-dicyclohexylcarbodiimide (DCC), N,N′-diisopropylcarbodiimide (DIPCI), 1-ethyl-3-(3-dimethylaminopropyl)carbodiimide (WSCI), 1-ethyl-3-(3-dimethylaminopropyl)carbodiimide hydrochloride (WSC·HCl), diethyl phosphorocyanidate (DEPC), benzotriazol-1-yloxy-tris(dimethylamino)phosphonium hexafluorophosphate (BOP), benzotriazol-1-yloxy-tris(pyrrolidinylamino)phosphonium hexafluorophosphate (PyBOP), 2-(1H-benzotriazol-1-yl)-1,1,3,3-tetramethyluronium tetrafluoroborate (TBTU), or the like. The reaction condition may vary depending on the reaction materials used. However, the reaction is generally carried out at −20° C. to 100° C., preferably 0° C. to 40° C. for 5 minutes to 30 hours, and preferably 2 hours to 20 hours to obtain the carboxylic acid compound (X). In addition, it is also possible to obtain the compound (X) by converting first the carboxylic acid compound (VIII) to an acid halide and reacting the acid halide with the amine compound (IX).

Further, among the compounds represented by the formula (V), the compound represented by the formula (XIV) can be produced according to the method described below, but not limited thereto.

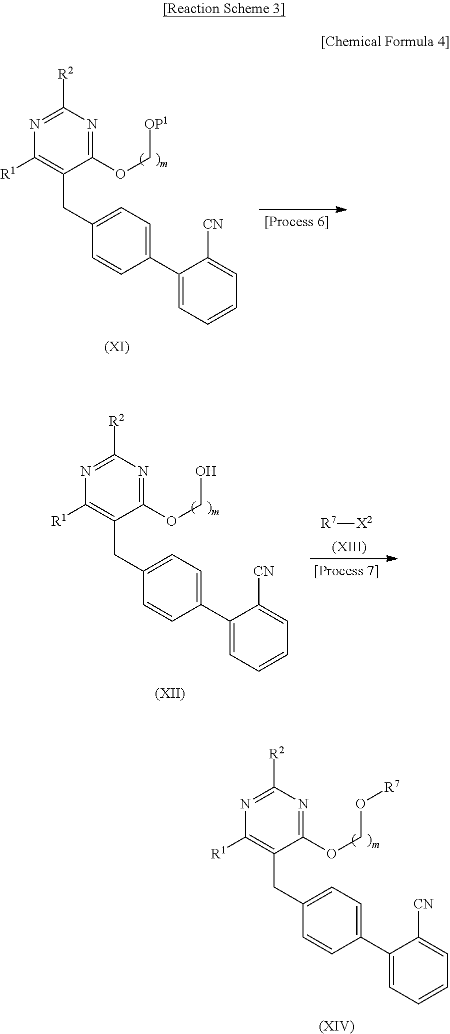

[in the formula, R¹ and R² are as defined above, R⁷ represents a C₁₋₆ alkyl group which may have a substituent, P¹ represents a protective group for a hydroxy group, X² represents a halogen atom, and m represents an integer of from 1 to 6].

### [Process 6]

The compound (XII) can be obtained by carrying out deprotection of P¹, which is a protective group for the pyrimidine derivative (XI). Method for deprotection is not specifically limited, and it can be carried out according to the method that is generally used as deprotection of the corresponding protective group (Protective Groups in Organic Synthesis Fourth Edition, John Wiley & Sons, Inc.).

### [Process 7]

The reaction between the compound (XII) and the halide (XIII) can be carried out in a solvent in the presence or absence of a base. The solvent is not specifically limited, and tetrahydrofuran, toluene, dioxane, N,N-dimethylformamide, N-methylpyrrolidone, dichloromethane, chloroform, acetonitrile, propionitrile, or the like may be used either alone or in combination thereof. The base is not specifically limited, and examples thereof include pyridine, N,N-dimethylaminopyridine (DMAP), collidine, lutidine, 1,8-diazabicyclo[5.4.0]-7-undecene (DBU), 1,5-diazabicyclo[4.3.0]-5-nonene (DBN), 1,4-diazabicyclo[2.2.2]octane (DABCO), triethylamine, diisopropylamine, diisopropylethylamine, diisopropylpentylamine, trimethylamine, lithium hydride, sodium hydride, potassium hydride, lithium hydroxide, sodium hydroxide, potassium hydroxide, lithium carbonate, sodium carbonate, potassium carbonate, cesium carbonate, sodium hydrogen carbonate, or the like. The reaction condition may vary depending on the reaction materials used. However, the reaction is generally carried out at −20 to 100° C., preferably 15 to 80° C. for 5 minutes to 36 hours, and preferably 5 hours to 24 hours to obtain the compound (XIV).

Further, among the compounds represented by the formula (I), the compound represented by the formula (Ib) can be produced according to the method described below, but not limited thereto.

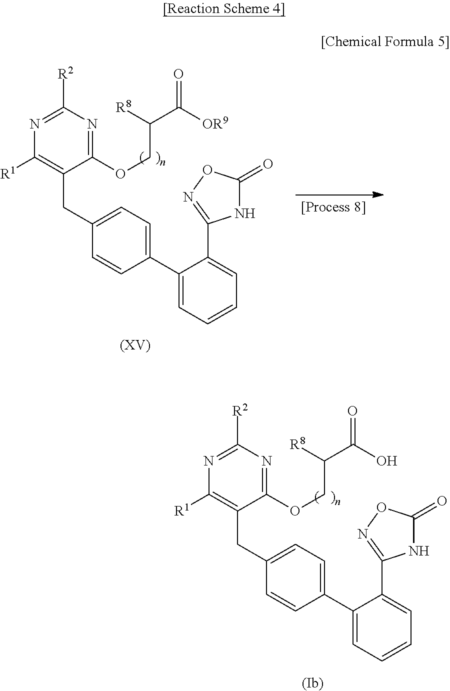

[in the formula, R¹ and R² are as defined above, R⁸ represents a hydrogen atom, a C₆₋₁₀ alkyl aryl group or a C₆₋₁₀ aryl C₁₋₆ alkyl group, R⁹ represents a C₁₋₆ alkyl group, and n represents an integer of from 1 to 6].

### [Process 8]

The carboxylic acid compound (Ib) is obtained by general hydrolysis of the carboxylic acid derivative (XV). The reaction can be carried out in a solvent in the presence of a base or an acid. The solvent is not specifically limited, and tetrahydrofuran, dioxane, methanol, ethanol, water, or the like may be used either alone or in combination thereof. The base is not specifically limited, and examples thereof include sodium hydroxide, potassium hydroxide, lithium carbonate, sodium carbonate, potassium carbonate, cesium carbonate, trimethylsilyloxy potassium, and the like. The acid is not specifically limited, and examples thereof include hydrochloric acid, acetic acid, trifluoroacetic acid, boron tribromide, aluminum chloride, and the like. The reaction condition may vary depending on the reaction materials used. However, the reaction is generally carried out at −20° C. to 100° C., preferably 15° C. to 80° C. for 5 minutes to 1 day, and preferably for 30 minutes to 13 hours to obtain the carboxylic acid compound (Ib).

Further, among the compounds represented by the formula (I), the compound represented by the formula (Ic) can be produced according to the method described below, but not limited thereto.

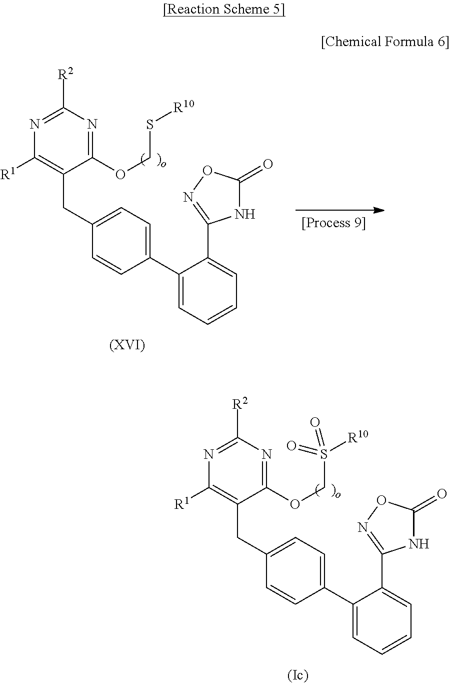

[in the formula, R¹ and R² are as defined above, R¹⁰ represents a C₁₋₆ alkyl group, and o represents an integer of from 1 to 6].

### [Process 9]

For oxidation of the compound (XVI), a common method for converting a sulfur atom to a sulfonyl group can be applied. For example, an oxidation reaction using hydrogen peroxide and a catalytic amount of sodium tungstate, molybdenum dichloride dioxide, or tantalum pentachloride can be employed, or sodium periodate, potassium periodate, metachloro perbenzoate (mCPBA), pyridinium chlorochromate (PCC), pyridinium dichromate (PDC), N-chlorosuccinimide (NCS), N-bromosuccinimide (NBS), N-iodosuccinimide (NIS), iodine, bromine, and the like can be used. The solvent is not specifically limited, and water, methanol, ethanol, isopropanol, acetonitrile, acetone, tetrahydrofuran, dichloromethane, chloroform, 1,2-dichloroethane, carbon tetrachloride, N,N-dimethylformamide, acetic acid, or the like may be used either alone or in combination thereof. The reaction condition may vary depending on the reaction materials used. However, the reaction is generally carried out at −20° C. to 100° C., preferably 15° C. to 80° C. for 5 minutes to 1 day, and preferably for 30 minutes to 13 hours to obtain the carboxylic acid compound (Ic).

Further, among the compounds represented by the formula (I), the compound represented by the formula (Id) can be produced according to the method described below, but not limited thereto.

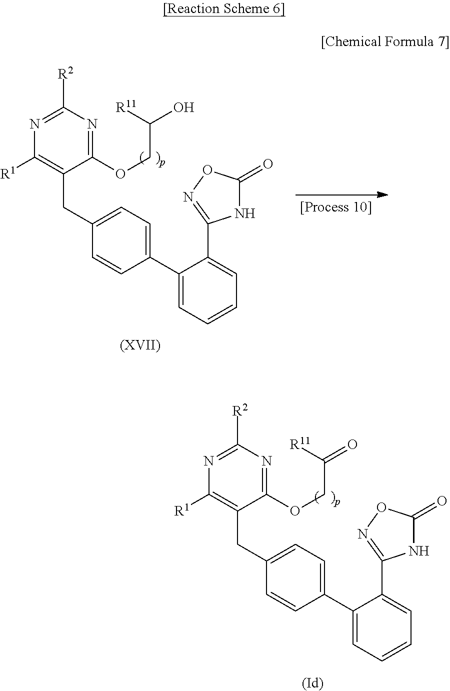

[in the formula, R¹ and R² are as defined above, R¹¹ represents a C₁₋₆ alkyl group, and p represents an integer of from 1 to 6].

### [Process 10]

For oxidation of the compound (XVII) to the compound (Id), a common method for oxidizing a hydroxy group to a ketone can be applied. For example, an oxidation condition such as Swern oxidation, Moffat oxidation, Dess-Martin oxidation, or the like, or PCC, PDC, manganese dioxide, tetrapropylammonium perruthenate (TPAP), or the like can be used. The solvent is not specifically limited, and tetrahydrofuran, dichloromethane, chloroform, 1,2-dichloroethane, N,N-dimethylformamide, or the like maybe used either alone or in combination thereof. The reaction condition may vary depending on the reaction materials used. However, the reaction is generally carried out at 0 to 180° C., preferably 20 to 100° C. for 1 minute to 2 weeks, and preferably 1 hour to 3 days to obtain the compound (Id).

Further, among the compounds represented by the formula (I), the compound represented by the formula (Ie) can be produced according to the method described below, but not limited thereto.

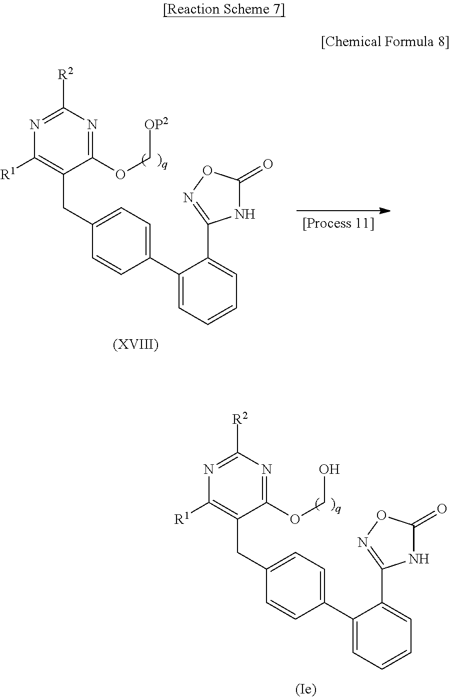

[in the formula, R¹ and R² are as defined above, P² represents a protective group for a hydroxy group, and q represents an integer of from 1 to 6].

### [Process 11]

The compound (Ie) can be obtained by carrying out deprotection of P², which is a protective group for the pyrimidine derivative (XVI). Method for deprotection is not specifically limited, and it can be carried out according to the method that is generally used as deprotection of the corresponding protective group (Protective Groups in Organic Synthesis Fourth Edition, John Wiley & Sons, Inc.).

If necessary, the intermediates and target compounds that are obtained from each of the reaction above can be isolated and purified by a purification method that is generally used in a field of organic synthesis chemistry, e.g., filtration, extraction, washing, drying, concentration, recrystallization, various chromatographic methods, and the like. Furthermore, the intermediates maybe used for the next reaction without any specific purification.

Various isomers may be isolated by applying a general method based on a difference in physicochemical properties among the isomers. For example, a racemic mixture may be resolved into an optically pure isomer by common racemic resolution like optical resolution by which a diastereomer salt is formed with a common optically active acid like tartaric acid or a method of using optically active chromatography. Further, a mixture of diastereomers can be resolved by fractional crystallization or various chromatographic methods, for example. Furthermore, an optically active compound can be also produced by using an appropriate starting compound that is optically active.

The compound (I) obtained may be converted into a salt according to a common method. Furthermore, it may be converted into a solvate with a solvent like a solvent for reaction or a solvent for recrystallization, or into a hydrate.

Examples of dosage form of the pharmaceutical agent containing the compounds of the invention, salts or solvates thereof as an effective component include, for example, those for oral administration such as tablet, capsule, granule, powder, syrup, or the like and those for parenteral administration such as intravenous injection, intramuscular injection, suppository, inhalant, transdermal preparation, eye drop, nasal drop, or the like. In order to prepare a pharmaceutical preparation in the various dosage forms, the effective component may be used alone, or may be used in appropriate combination with other pharmaceutically acceptable carriers such as excipients, binders, extending agents, disintegrating agents, surfactants, lubricants, dispersing agents, buffering agents, preservatives, corrigents, perfumes, coating agents, diluents, and the like to give a pharmaceutical composition.

Although the administration amount of the pharmaceutical agent of the invention may vary depending on the weight, age, sex, symptoms, and the like of a patient, in terms of the compound represented by the formula (I), generally 0.1 to 1000 mg, especially 1 to 300 mg, may be administered orally or parenterally at one time or several times as divided portions per day for an adult.

### EXAMPLES

Herein below, the invention will be explained in greater detail with reference to examples. However, the invention is not limited to these examples. The abbreviations used in the examples have the following meanings.

s: singlet

d: doublet

t: triplet

q: quartet

m: multiplet

br: broad

J: coupling constant

Hz: hertz

CDCl₃: deuterated chloroform

DMSO-d₆: deuterated dimethylsulfoxide

¹H-NMR: proton nuclear magnetic resonance

IR: infrared absorption spectrum

### Example 1

Production of ethyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetate

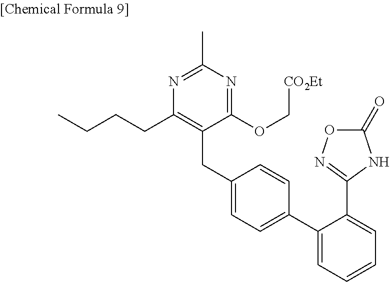

Process 1: 55% sodium hydride (31 mg, 0.72 mmol) was added to a N,N-dimethylformamide (3 mL) solution of 4′-[(4-butyl-2-methyl-6-oxo-1,6-dihydropyrimidin-5-yl)methyl]-[1,1′-biphenyl]-2-carbonitrile (214 mg, 0.60 mmol) and stirred for 30 minutes at room temperature. The reaction solution was added ethyl bromoacetate (110 mg, 0.66 mmol) and stirred overnight at 70° C. The reaction mixture was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=2:1) to obtain ethyl 2-{{6-butyl-5-[(2′-cyano-[1,1′-biphenyl]-4-yl)methyl]-2-methylpyrimidin-4-yl}oxy}acetate (80 mg, 30%) as yellow oil.

¹H-NMR (CDCl₃) δ:
0.87 (3H, t, J=7 Hz), 1.27 (3H, t, J=7 Hz),
1.30-1.40 (2H, m), 1.47-1.59 (2H, m), 2.54 (3H, s),
2.65-2.74 (2H, m), 4.09 (2H, s), 4.24 (2H, q, J=7 Hz),
4.93 (2H, s), 7.32 (2H, d, J=8 Hz), 7.38-7.50 (4H, m),
7.58-7.65 (1H, m), 7.74 (1H, d, J=8 Hz).

Process 2: Sodium hydrogen carbonate (307 mg, 3.7 mmol) was added to a dimethylsulfoxide solution (2 mL) of hydroxylamine hydrochloride (212 mg, 3.0 mmol) and stirred for 1 hour at 40° C. The reaction mixture was added dimethylsulfoxide solution (1 mL) of ethyl 2-{{6-butyl-5-[(2′-cyano-[1,1′-biphenyl]-4-yl)methyl]-2-methylpyrimidin-4-yl}oxy}acetate (54 mg, 0.12 mmol) and stirred overnight at 90° C. The reaction solution was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=2:1) to obtain ethyl 2-{{6-butyl-5-{[2′-(N′-hydroxylcarbamimidoyl)-[1,1′-biphenyl]-4-yl]methyl}-2-methylpyrimidin-4-yl}oxy}acetate.

Process 3: 1,1′-carbonyldiimidazole (7 mg, 0.04 mmol) and 1,8-diazabicyclo[5.4.0]undec-7-ene (6 mg, 0.04 mmol) were added to a N,N-dimethylformamide solution (1 mL) of ethyl 2-{{6-butyl-5-{[2′-(N′-hydroxylcarbamimidoyl)-[1,1′-biphenyl]-4-yl]methyl}-2-methylpyrimidin-4-yl}oxy}acetate (7 mg, 0.02 mmol) and stirred for 1 hour at room temperature. After completion of the reaction, the reaction mixture was added water and extracted with ethyl acetate. The organic layer was combined, washed with water and brine, dried over anhydrous sodium sulfate, and concentrated in vacuo. The residues obtained were purified by preparative thin layer chromatography (ethyl acetate) to obtain ethyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetate (5 mg, two-step yield; 59%) as yellow oil.

¹H-NMR (CDCl₃) δ:
0.92 (3H, t, J=7.2 Hz), 1.24 (3H, t, J=7.2 Hz),
1.35-1.45 (2H, m), 1.55-1.68 (2H, m), 2.52 (3H, s),
2.69-2.78 (2H, m), 4.04 (2H, s),
4.14 (2H, q, J=7.2 Hz), 4.89 (2H, s), 7.24 (4H, brs),
7.39-7.65 (3H, m), 7.87 (1H, d, J=7.8 Hz).

### Example 2

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetic acid

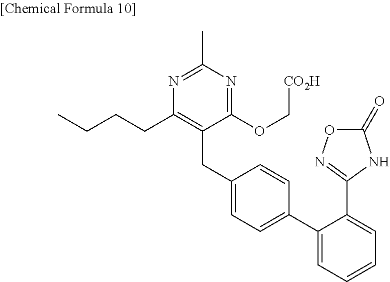

Process 1: Lithium hydroxide monohydrate (237 mg, 5.6 mmol) was added to a methanol (10 mL) and water (5 mL) mixture solution of ethyl 2-{{6-butyl-5-[(2′-cyano-[1,1′-biphenyl]-4-yl)methyl]-2-methylpyrimidin-4-yl}oxy}acetate (500 mg, 1.1 mmol) and stirred for 1 hour at room temperature. The reaction mixture was added water and extracted with chloroform. The organic layer was combined, washed with water and brine, dried over anhydrous sodium sulfate, and concentrated in vacuo. The residues obtained were subjected to silica gel column chromatography (chloroform/methanol=20:1) to obtain 2-{{6-butyl-5-[(2′-cyano-[1,1′-biphenyl]-4-yl)methyl]-2-methylpyrimidin-4-yl}oxy}acetic acid (467 mg, 100%) as a white solid.

¹H-NMR (CD₃OD) δ:
0.86 (3H, t, J=7 Hz), 1.29-1.48 (4H, m), 2.58 (3H, s),
2.71-2.78 (2H, m), 4.17 (2H, s), 5.06 (2H, s),
7.40 (2H, d, J=8 Hz), 7.45-7.57 (4H, m),
7.66-7.74 (1H, m), 7.81 (1H, d, J=8 Hz).

### Process 2:

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetic acid was obtained as a pale yellow solid (two-step yield; 87%) according to the same reaction and treatment as the Processes 2 and 3 of the Example 1 by using 2-{{6-butyl-5-[(2′-cyano-[1,1′-biphenyl]-4-yl)methyl]-2-methylpyrimidin-4-yl}oxy}acetic acid instead of the ethyl 2-{{6-butyl-5-[(2′-cyano-[1,1′-biphenyl]-4-yl)methyl]-2-methylpyrimidin-4-yl}oxy}acetate.

¹H-NMR (CD₃OD) δ:
0.93 (3H, t, J=7.3 Hz), 1.40-1.52 (2H, m),
1.66-1.78 (2H, m), 2.82 (3H, s), 3.04-3.18 (2H, m),
4.05 (2H, s), 5.05 (2H, s), 7.14 (2H, d, J=7.6 Hz),
7.21 (2H, d, J=7.6 Hz), 7.36 (1H, d, J=7.2 Hz),
7.45-7.65 (2H, m), 7.90 (1H, d, J=8.0 Hz).

### Example 3

Production of 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetamide

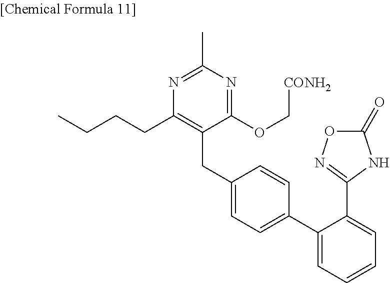

Process 1: A dichloromethane (3 mL) solution of 2-{{6-butyl-5-[(2′-cyano-[1,1′-biphenyl]-4-yl)methyl]-2-methylpyrimidin-4-yl}oxy}acetic acid (83 mg, 0.2 mmol) and 1-hydroxybenzotriazole hydrate (42 mg, 0.3 mmol) was stirred for 1 hour at room temperature. Subsequently, ammonia (28% aqueous solution, 0.4 mL) was added and stirred for 4 hours at room temperature. The reaction mixture was added water and extracted with chloroform. The organic layer was combined, washed with water and brine, dried over anhydrous sodium sulfate, and concentrated in vacuo. The residues obtained were subjected to silica gel column chromatography (chloroform/methanol=20:1) to obtain 2-{{6-butyl-5-[(2′-cyano-[1,1′-biphenyl]-4-yl)methyl]-2-methylpyrimidin-4-yl}oxy}acetamide (73 mg, 88%) as a pale yellow solid.

¹H-NMR (CDCl₃) δ:
0.94 (3H, t, J=7 Hz), 1.37-1.49 (2H, m),
1.63-1.76 (2H, m), 2.59 (3H, s), 2.79-2.89 (2H, m),
4.08 (2H, s), 4.87 (2H, s), 5.28 (1H, brs), 5.48 (1H, brs),
7.25 (2H, d, J=8 Hz), 7.43-7.53 (4H, m), 7.62-7.79 (2H, m).

Process 2:

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetamide was obtained as a pale yellow oil (two-step yield; 44%) according to the same reaction and treatment as the Processes 2 and 3 of the Example 1 by using 2-{{6-butyl-5-[(2′-cyano-[1,1′-biphenyl]-4-yl)methyl]-2-methylpyrimidin-4-yl}oxy}acetamide instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.92 (3H, t, J=7.2 Hz), 1.35-1.47 (2H, m),
1.60-1.72 (2H, m), 2.54 (3H, s), 2.73-2.88 (2H, m),
3.99 (2H, s), 4.75 (2H, s), 5.41 (1H, brs), 6.71 (1H, brs),
7.06 (2H, d, J=8.2 Hz), 7.20 (2H, d, J=8.2 Hz),
7.29-7.39 (2H, m), 7.44-7.57 (2H, m).

### Example 4

Production of 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylacetamide

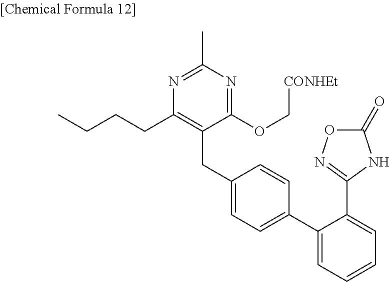

Process 1:

2-{{6-butyl-5-[(2′-cyano-[1,1′-biphenyl]-4-yl)methyl]-2-methylpyrimidin-4-yl}oxy}-N-ethylacetamide was obtained as a pale yellow solid (yield 81%) according to the same reaction and treatment as the Process 1 of the Example 3 by using ethylamine instead of the ammonia.

¹H-NMR (CDCl₃) δ:
0.92 (6H, t, J=7 Hz), 1.35-1.46 (2H, m), 1.60-1.69 (2H, m),
2.59 (3H, s), 2.75-2.83 (2H, m), 3.08-3.17 (2H, m),
4.10 (2H, s), 4.87 (2H, s), 5.75 (1H, brs),
7.25 (2H, d, J=8 Hz), 7.43-7.56 (4H, m), 7.62-7.69 (1H, m),
7.77 (1H, d, J=8 Hz).

Process 2:

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethyl acetamide was obtained as a pale yellow solid (two-step yield; 24%) according to the same reaction and treatment as the Processes 2 and 3 of the Example 1 by using 2-{{6-butyl-5-[(2′-cyano-[1,1′-biphenyl]-4-yl)methyl]-2-methylpyrimidin-4-yl}oxy}-N-ethyl acetamide instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.92 (3H, t, J=7.2 Hz), 0.96 (3H, t, J=7.2 Hz),
1.35-1.46 (2H, m), 1.57-1.69 (2H, m), 2.49 (3H, s),
2.64-2.74 (2H, m), 3.07-3.15 (2H, m), 4.02 (2H, s),
4.75 (2H, s), 5.84 (1H, brs), 7.12 (2H, d, J=7.6 Hz),
7.24 (2H, d, J=7.6 Hz), 7.35-7.41 (1H, m),
7.46 (1H, d, J=8.0 Hz), 7.86 (1H, t, J=7.2 Hz),
7.74 (1H, d, J=7.2 Hz).

### Example 5

Production of 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylacetamide

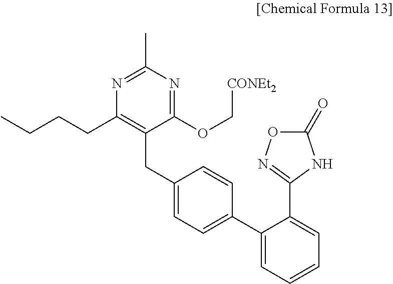

Process 1:

2-{{6-butyl-5-[(2′-cyano-[1,1′-biphenyl]-4-yl)-2-methylpyrimidin-4-yl]oxy}-N,N-diethylacetamide was obtained as a pale yellow solid (yield 72%) according to the same reaction and treatment as the Process 1 of the Example 3 by using diethylamine instead of the ammonia.

¹H-NMR (CDCl₃) δ:
0.87 (3H, t, J=7 Hz), 1.15-1.41 (8H, m), 1.48-1.58 (2H, m),
2.53 (3H, s), 2.64-2.73 (2H, m), 3.29-3.46 (4H, m),
4.12 (2H, s), 5.02 (2H, s), 7.33-7.50 (6H, m),
7.58-7.64 (1H, m), 7.73 (1H, d, J=8 Hz).

Process 2:

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylacetamide was obtained as a pale yellow oil (two-step yield; 40%) according to the same reaction and treatment as the Processes 2 and 3 of the Example 1 by using 2-{{6-butyl-5-[(2′-cyano-[1,1′-biphenyl]-4-yl)-2-methylpyrimidin-4-yl]oxy}-N,N-diethylacetamide instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.88 (3H, t, J=7.2 Hz), 1.09 (3H, t, J=7.2 Hz),
1.28-1.40 (5H, m), 1.52-1.64 (2H, m), 2.52 (3H, s),
2.62-2.71 (2H, m), 3.31-3.42 (4H, m), 4.01 (2H, s),
b 4.95 (2H, s), 7.07 (2H, d, J=8.0 Hz),
7.13 (2H, d, J=8.0 Hz), 7.36-7.57 (3H, m),
7.74 (1H, d, J=7.6 Hz).

### Example 6

Production of ethyl 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoate

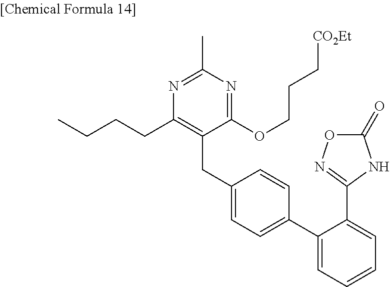

Process 1: Ethyl 4-{{6-butyl-5-[(2′-cyano-[1,1′-biphenyl]-4-yl)methyl]-2-methylpyrimidin-4-yl}oxy}butanoate was obtained as a pale yellow oil (yield 43%) according to the same reaction and treatment as the Process 1 of the Example 1 by using ethyl bromobutanoate instead of the ethyl bromoacetate.

¹H-NMR (CDCl₃) δ:
0.90 (3H, t, J=7 Hz), 1.18 (3H, t, J=7 Hz),
1.31-1.43 (2H, m), 1.53-1.64 (2H, m), 1.97-2.07 (2H, m),
2.28 (2H, t, J=7 Hz), 2.57 (3H, s), 2.68-2.76 (2H, m),
4.01 (2H, s), 4.07 (2H, q, J=7 Hz), 4.39 (2H, t, J=6 Hz),
7.24 (2H, d, J=8 Hz), 7.37-7.52 (4H, m),
7.58-7.65 (1H, m), 7.74 (1H, d, J=8 Hz).

Process 2: Ethyl 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoate was obtained as a pale yellow oil (two step yield; 32%) according to the same reaction and treatment as the Processes 2 and 3 of the Example 1 by using ethyl 4-{{6-butyl-5-[(2′-cyano-[1,1′-biphenyl]-4-yl)methyl]-2-methylpyrimidin-4-yl}oxy}butanoate instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.91 (3H, t, J=7.2 Hz), 1.15 (3H, t, J=7.2 Hz),
1.33-1.44 (2H, m), 1.56-1.68 (2H, m), 1.83-1.94 (2H, m),
2.00-2.09 (2H, m), 2.55 (3H, s), 2.65-2.78 (2H, m),
3.97 (2H, s), 4.01 (2H, q, J=7.2 Hz),
4.30 (2H, t, J=5.6 Hz), 7.09 (2H, d, J=8.0 Hz),
7.25 (2H, d, J=8.0 Hz), 7.38-7.64 (3H, m),
7.75 (1H, d, J=7.6 Hz).

### Example 7

Production of 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoic acid

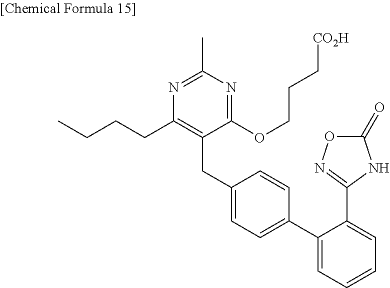

Process 1:

4-{{6-butyl-5-[(2′-cyano-[1,1′-biphenyl]-4-yl)methyl]-2-methylpyrimidin-4-yl}oxy}butanoic acid was obtained as a white solid (yield 100%) according to the same reaction and treatment as the Process 1 of the Example 2 by using ethyl 4-{{6-butyl-5-[(2′-cyano-[1,1′-biphenyl]-4-yl)methyl]-2-methylpyrimidin-4-yl}oxy}butanoate instead of the ethyl 2-{{6-butyl-5-[(2′-cyano-[1,1′-biphenyl]-4-yl)methyl]-2-methylpyrimidin-4-yl}oxy}acetate.

¹H-NMR (CD₃OD) δ:
0.90 (3H, t, J=7 Hz), 1.35-1.46 (2H, m), 1.51-1.63 (2H, m),
1.97-2.06 (2H, m), 2.23-2.31 (2H, m), 2.68 (3H, s),
2.84-2.93 (2H, m), 4.12 (2H, s), 4.54 (2H, t, J=6 Hz),
7.25-7.34 (2H, m), 7.46-7.57 (4H, m), 7.62-7.74 (1H, m),
7.77 (1H, d, J=8 Hz).

Process 2:

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoic acid was obtained as a pale yellow oil (two-step yield; 87%) according to the same reaction and treatment as the Processes 2 and 3 of the Example 1 by using 4-{{6-butyl-5-[(2′-cyano-[1,1′-biphenyl]-4-yl)methyl]-2-methylpyrimidin-4-yl}oxy}butanoic acid instead of the ethyl 2-{{6-butyl-5-[(2′-cyano-[1,1′-biphenyl]-4-yl)methyl]-2-methylpyrimidin-4-yl}oxy}acetate.

¹H-NMR (CD₃OD) δ:

0.92 (3H, t, J=7.3 Hz), 1.33-1.46 (2H, m), 1.51-1.61 (2H, m),
1.96-2.06 (2H, m), 2.26-2.34 (2H, m), 2.62 (3H, s),
2.76-2.84 (2H, m), 4.07 (2H, s), 4.49 (2H, t, J=6.0 Hz),
7.20 (2H, d, J=8.4 Hz), 7.27 (2H, d, J=8.4 Hz),
7.48-7.55 (2H, m), 7.60-7.68 (2H, m).

### Example 8

Production of 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-butanamide

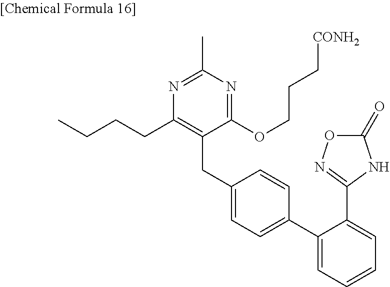

Process 1:

4-{{6-butyl-5-[(2′-cyano-[1,1′-biphenyl]-4-yl)methyl]-2-methylpyrimidin-4-yl}oxy}butanamide was obtained as a pale yellow solid (yield 80%) according to the same reaction and treatment as the Process 1 of the Example 3 by using 4-{{6-butyl-5-[(2′-cyano-[1,1′-biphenyl]-4-yl)methyl]-2-methylpyrimidin-4-yl}oxy}butanoic acid instead of the 2-{{6-butyl-5-[(2′-cyano-[1,1′-biphenyl]-4-yl)methyl]-2-methylpyrimidin-4-yl}oxy}acetic acid.

¹H-NMR (CDCl₃) δ:
0.91 (3H, t, J=7 Hz), 1.32-1.44 (2H, m), 1.56-1.67 (2H, m),
1.97-2.16 (4H, m), 2.57 (3H, s), 2.70-2.77 (2H, m),
4.02 (2H, s), 4.36 (2H, t, J=6 Hz), 5.44 (1H, brs),
5.66 (1H, brs), 7.22 (2H, d, J=8 Hz), 7.42-7.53 (4H, m),
7.61-7.67 (1H, m), 7.74-7.77 (1H, m).

Process 2:

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-butanamide was obtained as a pale yellow oil (two-step yield; 37%) according to the same reaction and treatment as the Processes 2 and 3 of the Example 1 by using 4-{{6-butyl-5-[(2′-cyano-[1,1′-biphenyl]-4-yl)methyl]-2-methylpyrimidin-4-yl}oxy}butanamide instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.89 (3H, t, J=7.2 Hz), 1.32-1.44 (2H, m),
1.58-2.00 (6H, m), 2.55 (3H, s), 2.66-2.74 (2H, m),
3.91 (2H, s), 4.20 (2H, brs), 5.28 (1H, brs), 6.49 (1H, brs),
6.94 (2H, d, J=7.6 Hz), 7.17 (2H, d, J=7.6 Hz),
7.25-7.44 (3H, m), 7.51-7.58 (1H, m).

### Example 9

Production of 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylbutanamide

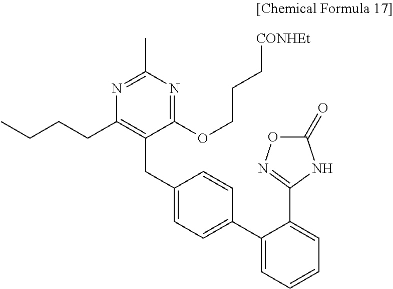

Process 1:

4-{{6-butyl-5-[(2′-cyano-[1,1′-biphenyl]-4-yl)methyl]-2-methylpyrimidin-4-yl}oxy}-N-ethylbutanamide was obtained as a pale yellow solid (yield 89%) according to the same reaction and treatment as the Process 1 of the Example 4 by using 4-{{6-butyl-5-[(2′-cyano-[1,1′-biphenyl]-4-yl)methyl]-2-methylpyrimidin-4-yl}oxy}butanoic acid instead of the 2-{{6-butyl-5-[(2′-cyano-[1,1′-biphenyl]-4-yl)methyl]-2-methylpyrimidin-4-yl}oxy}acetic acid.

¹H-NMR (CDCl₃) δ:
0.91 (3H, t, J=7 Hz), 1.01 (3H, t, J=7 Hz),
1.34-1.44 (2H, m), 1.57-1.67 (2H, m), 1.99-2.08 (4H, m),
2.57 (3H, s), 2.70-2.78 (2H, m), 3.13-3.22 (2H, m),
4.01 (2H, s), 4.32-4.38 (2H, m), 5.61 (1H, brs),
7.22 (2H, d, J=8 Hz), 7.41-7.52 (4H, m), 7.61-7.68 (1H, m),
7.50 (1H, d, J=7 Hz).

Process 2:

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylbutanamide was obtained as a pale yellow oil (two-step yield; 28%) according to the same reaction and treatment as the Processes 2 and 3 of the Example 1 by using 4-{{6-butyl-5-[(2′-cyano-[1,1′-biphenyl]-4-yl)methyl]-2-methylpyrimidin-4-yl}oxy}-N-ethylbutanamide instead of the ethyl 2-{{6-butyl-5-[(2′-cyano-[1,1′-biphenyl]-4-yl)methyl]-2-methylpyrimidin-4-yl}oxy} acetate.

¹H-NMR (CDCl₃) δ:
0.92 (3H, t, J=7.2 Hz), 1.03 (3H, t, J=7.2 Hz),
1.35-1.46 (2H, m), 1.62-1.85 (6H, m), 2.58 (3H, s),
2.74-2.80 (2H, m), 3.07-3.16 (2H, m), 3.95 (2H, s),
4.30 (2H, t, J=5.6 Hz), 5.55 (1H, brs),
7.05 (2H, d, J=7.6 Hz), 7.27 (2H, d, J=7.6 Hz),
7.40-7.48 (2H, m), 7.55-7.62 (1H, m),
7.69 (1H, d, J=8.0 Hz).

### Example 10

Production of 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylbutanamide]

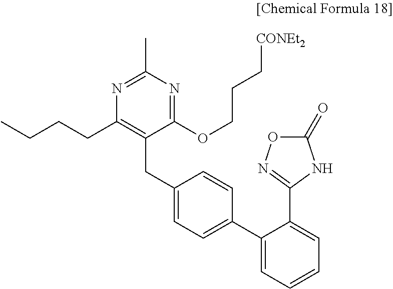

Process 1:

4-{{6-butyl-5-[(2′-cyano-[1,1′-biphenyl]-4-yl)methyl]-2-methylpyrimidin-4-yl}oxy}-N,N-diethylbutanamide was obtained as a pale yellow solid (yield 91%) according to the same reaction and treatment as the Process 1 of the Example 5 by using 4-{{6-butyl-5-[(2′-cyano-[1,1′-biphenyl]-4-yl)methyl]-2-methylpyrimidin-4-yl}oxy}butanoic acid instead of the 2-{{6-butyl-5-[(2′-cyano-[1,1′-biphenyl]-4-yl)methyl]-2-methylpyrimidin-4-yl}oxy}acetic acid.

¹H-NMR (CDCl₃) δ:
0.88 (3H, t, J=7 Hz), 1.01-1.11 (6H, m), 1.31-1.42 (2H, m),
1.51-1.62 (2H, m), 2.06-2.15 (2H, m), 2.32-2.39 (2H, m),
2.57 (3H, s), 2.65-2.72 (2H, m), 3.16 (2H, q, J=7 Hz),
3.34 (2H, q, J=7 Hz), 4.02 (2H, s), 4.40-4.43 (2H, m),
7.24 (2H, d, J=8 Hz), 7.39-7.50 (4H, m), 7.58-7.66 (1H, m),
7.74 (1H, d, J=8 Hz).

Process 2:

4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylbutanamide was obtained as a pale yellow oil (two-step yield; 30%) according to the same reaction and treatment as the Processes 2 and 3 of the Example 1 by using 4-{{6-butyl-5-[(2′-cyano-[1,1′-biphenyl]-4-yl)methyl]-2-methylpyrimidin-4-yl}oxy}-N,N-diethylbutanamide instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.88-0.92 (6H, m), 1.02-1.16 (3H, m), 1.31-1.44 (2H, m),
1.52-1.68 (4H, m), 2.09-2.20 (2H, m), 2.56 (3H, s),
2.65-2.77 (2H, m), 3.06-3.26 (4H, m), 3.92 (2H, s),
4.33 (2H, brs), 7.01 (2H, d, J=7.6 Hz),
7.19 (2H, d, J=7.6 Hz), 7.29-7.54 (3H, m),
7.65 (1H, d, J=7.6 Hz).

### Example 11

Production of 3-{4′-{[4-butyl-6-(2-ethoxyethoxy)-2-methylpyrimidin-5-yl]methyl}-[1,1′-biphenyl]-2-yl}-1,2,4-oxadiazol-5(4H)-one

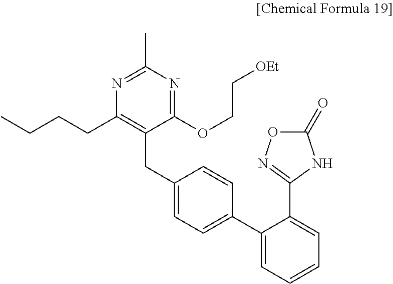

Process 1:

4′-{[4-butyl-6-(2-ethoxyethoxy)-2-methylpyrimidin-5-yl]methyl}-[1,1′-biphenyl]-2-carbonitrile was obtained as a pale yellow oil (yield 91%) according to the same reaction and treatment as the Process 1 of the Example 1 by using 1-bromo-2-ethoxyethane instead of the ethyl bromoacetate.

¹H-NMR (CDCl₃) δ:
0.89 (3H, t, J=7 Hz), 1.18 (3H, t, J=7 Hz),
1.32-1.42 (2H, m), 1.52-1.62 (2H, m), 2.57 (3H, s),
2.67-2.74 (2H, m), 3.52 (2H, t, J=7 Hz), 3.71-3.76 (2H, m),
4.03 (2H, s), 4.51-4.55 (2H, m), 7.27-7.36 (2H, m),
7.37-7.49 (4H, m), 7.57-7.64 (1H, m), 7.74 (1H, d, J=8 Hz).

Process 2:

3-{4′-{{4-butyl-6-(2-ethoxyethoxy)-2-methylpyrimidin-5-yl}methyl}-[1,1′-biphenyl]-2-yl}-1,2,4-oxadiazol-5(4H)-one was obtained as a pale yellow oil (two-step yield; 35%) according to the same reaction and treatment as the Processes 2 and 3 of the Example 1 by using 4′-{[4-butyl-6-(2-ethoxyethoxy)-2-methylpyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.90 (3H, t, J=7.2 Hz), 1.05 (3H, t, J=7.2 Hz),
1.33-1.42 (2H, m), 1.52-1.63 (2H, m), 2.47 (3H, s),
2.61-2.68 (2H, m), 3.44 (2H, t, J=7.2 Hz), 3.65-3.74 (2H, m),
3.97 (2H, s), 4.44-4.52 (2H, m), 7.16-7.22 (4H, m),
7.38-7.49 (2H, m), 7.56-7.64 (1H, m),7.79 (1H, d, J=7.6 Hz).

### Example 12

Production of 3-{4′-{[4-butyl-2-methyl-6-(2-propoxyethoxy)pyrimidin-5-yl]methyl}-[1,1′-biphenyl]-2-yl}-1,2,4-oxadiazol-5(4H)-one

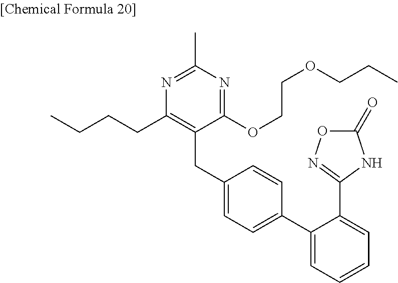

Process 1:

4′-{{4-butyl-6-{2-[(tert-butyldimethylsilyl)oxy]ethoxy}-2-methylpyrimidin-5-yl}methyl}-1-[1,1′-biphenyl]-2-carbonitrile was obtained as a pale yellow oil (yield 34%) according to the same reaction and treatment as the Process 1 of the Example 1 by using [(2-bromoethan-1-yl)oxy](tert-butyl)dimethylsilane instead of the ethyl bromoacetate.

¹H-NMR (CDCl₃) δ:
0.05 (6H, s), 0.86-0.90 (12H, m), 1.31-1.41 (2H, m),
1.51-1.62 (2H, m), 2.57 (3H, s), 2.65-2.72 (2H, m),
3.91 (2H, t, J=5 Hz), 4.03 (2H, s), 4.45 (2H, t, J=5 Hz),
7.26 (2H, d, J=8 Hz), 7.36-7.49 (4H, m),
7.57-7.64 (1H, m), 7.74 (1H, d, J=8 Hz).

Process 2: Tetrabutylammonium fluoride (1 M tetrahydrofuran solution, 1.5 mL) was added to a tetrahydrofuran (4 mL) solution of 4′-{{4-butyl-6-{2-[(tert-butyldimethylsilyl)oxy]ethoxy}-2-methylpyrimidin-5-yl}methyl}-1-[1,1′-biphenyl]-2-carbonitrile (200 mg, 0.3 mmol) and stirred overnight at room temperature. The reaction mixture was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=3:1) to obtain 4′-{[4-butyl-6-(2-hydroxyethoxy)-2-methylpyrimidin-5-yl]methyl}-[1,1′-biphenyl]-2-carbonitrile (75 mg, 63%) as a pale yellow oil (yield 63%).

¹H-NMR (CDCl₃) δ:
0.90 (3H, t, J=7 Hz), 1.33-1.43 (2H, m), 1.54-1.64 (2H, m),
2.58 (3H, s), 2.70-2.78 (2H, m), 3.27 (1H, brs),
3.82-3.87 (2H, m), 4.03 (2H, s), 4.47-4.52 (2H, m),
7.24 (2H, d, J=8 Hz), 7.39-7.50 (4H, m), 7.58-7.66 (1H, m),
7.74 (1H, d, J=8 Hz).

Process 3: 55% sodium hydride (26 mg, 0. 6 mmol) was added to a N,N-dimethylformamide (2 mL) solution of 4′-{[4-butyl-6-(2-hydroxyethoxy)-2-methylpyrimidin-5-yl]methyl}-[1,1′-biphenyl]-2-carbonitrile (80 mg, 0.20 mmol) and stirred for 30 minutes at room temperature. The reaction solution was then added n-iodopropane (102 mg, 0.6 mmol) and stirred overnight at 50° C. The reaction mixture was added water and extracted with ethyl acetate. The organic layer was combined, washed with water and brine, dried over anhydrous sodium sulfate, and concentrated in vacuo. The residues obtained were subjected to preparative thin layer chromatography (hexane/ethyl acetate=3:1) to obtain 4′-{[4-butyl-2-methyl-6-(2-propoxyethoxy)pyrimidin-5-yl]methyl}-[1,1′-biphenyl]-2-carbonitrile (41 mg, 46%) as colorless oil.

¹H-NMR (CDCl₃) δ:
0.85-0.96 (6H, m), 1.31-1.42 (2H, m), 1.51-1.62 (4H, m),
2.57 (3H, s), 2.67-2.74 (2H, m), 3.43 (2H, t, J=7 Hz),
3.71-3.76 (2H, m), 4.03 (2H, s), 4.49-4.55 (2H, m),
7.29 (2H, d, J=8 Hz), 7.38-7.49 (4H, m), 7.58-7.65 (1H, m),
7.74 (1H, d, J=8 Hz).

Process 4:

3-{4′-{[4-butyl-2-methyl-6-(2-propoxyethoxy)pyrimidin-5-yl]methyl}-[1,1′-biphenyl]-2-yl}-1,2,4-oxadiazol-5(4H)-one was obtained as a pale yellow oil (two-step yield; 53%) according to the same reaction and treatment as the Processes 2 and 3 of the Example 1 by using 4′-{[4-butyl-2-methyl-6-(2-propoxyethoxy)pyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.80 (3H, t, J=7.3 Hz), 0.91 (3H, t, J=7.2 Hz),
1.31-1.49 (4H, m), 1.51-1.64 (2H, m), 2.48 (3H, s),
2.61-2.69 (2H, m), 3.34 (2H, t, J=6.8 Hz), 3.67-3.72 (2H, m),
3.98 (2H, s), 4.43-4.49 (2H, m), 7.21 (4H, brs),
7.38-7.53 (2H, m), 7.56-7.65 (1H, m), 7.82 (1H, d, J=7.6 Hz).

### Example 13

Production of 3-{4′-{{4-butyl-6-[2-(2-methoxyethoxy)ethoxy]-2-methylpyrimidin-5-yl}methyl}-[1,1′-biphenyl]-2-yl}-1,2,4-oxadiazol-5(4H)-one

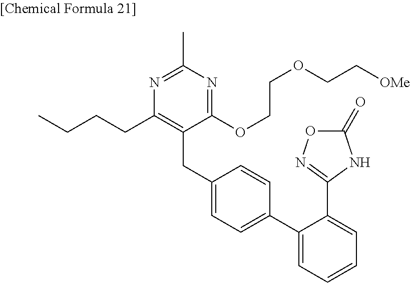

Process 1:

4′-{{4-butyl-6-[2-(2-methoxyethoxy)-2-methylpyrimidin-5-yl]methyl}-[1,1′-biphenyl]-2-carbonitrile was obtained as a pale yellow oil (yield 43%) according to the same reaction and treatment as the Process 3 of the Example 12 by using 1-bromo-2-methoxyethane instead of the propyl bromide.

¹H-NMR (CDCl₃) δ:
0.89 (3H, t, J=7 Hz), 1.32-1.42 (2H, m), 1.52-1.62 (2H, m),
2.56 (3H, s), 2.65-2.74 (2H, m), 3.35 (3H, s),
b 3.48-3.53 (2H, m), 3.60-3.65 (2H, m), 3.75-3.84 (2H, m),
4.03 (2H, s), 4.53-4.59 (2H, m), 7.28 (2H, d, J=8 Hz),
7.38-7.49 (4H, m), 7.57-7.65 (1H, m), 7.74 (1H, d, J=8 Hz).

Process 2:

3-{4′-{{4-butyl-6-[2-(2-methoxyethoxy)ethoxy]-2-methylpyrimidin-5-yl}methyl}-[1,1′-biphenyl]-2-yl}-1,2,4-oxadiazof-5 (4H)-one was obtained as a pale yellow oil (two-step yield; 43%) according to the same reaction and treatment as the Processes 2 and 3 of the Example 1 by using 4′-{{4-butyl-6-[2-(2-methoxyethoxy)-2-methylpyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.92 (3H, t, J=7.2 Hz), 1.34-1.46 (2H, m),
1.58-1.68 (2H, m), 2.55 (3H, s), 2.70-2.77 (2H, m),
2.91 (3H, s), 3.31-3.43 (4H, m), 3.66-3.72 (2H, m),
3.99 (2H, s), 4.43-4.47 (2H, m), 7.14 (2H, d, J=8.3 Hz),
7.23 (2H, d, J=8.3 Hz), 7.43-7.49 (2H, m),
7.60 (1H, dt, J=1.4, 7.6 Hz), 7.77 (1H, d, J=7.8 Hz).

### Example 14

Production of 3-{4′-{{4-[2-(benzyloxy)ethoxy]-6-butyl-2-methylpyrimidin-5-yl}methyl}-[1,1′-biphenyl]-2-yl}-1,2,4-oxadiazol-5(4H)-one

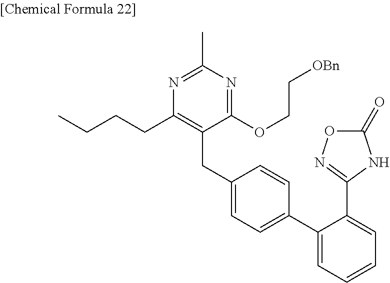

Process 1:

4′-{{4-[2-(benzyloxy)ethoxy]-6-butyl-2-methylpyrimidin-5-yl}methyl}-[1,1′-biphenyl]-2-carbonitrile was obtained as a pale yellow oil (yield 88%) according to the same reaction and treatment as the Process 3 of the Example 12 by using benzyl bromide instead of the propyl bromide.

¹H-NMR (CDCl₃) δ:
0.89 (3H, t, J=7 Hz), 1.32-1.44 (2H, m), 1.52-1.63 (2H, m),
2.55 (3H, s), 2.66-2.73 (2H, m), 3.78 (2H, t, J=5 Hz),
4.02 (2H, s), 4.53-4.59 (4H, m), 7.27-7.42 (11H, m),
7.56-7.76 (2H, m).

Process 2:

3-{4′-{{4-[2-(benzyloxy)ethoxy]-6-butyl-2-methylpyrimidin-5-yl}methyl}-[1,1′-biphenyl]-2-yl}-1,2,4-oxadiazol-5(4H)-one was obtained as a pale yellow oil (two-step yield; 28%) according to the same reaction and treatment as the Processes 2 and 3 of the Example 1 by using 4′-{{4-[2-(benzyloxy)ethoxy]-6-butyl-2-methylpyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.90 (3H, t, J=7.2 Hz), 1.31-1.42 (2H, m),
1.52-1.64 (2H, m), 2.45 (3H, s), 2.58-2.67 (2H, m),
3.76 (2H, t, J=4.4 Hz), 3.97 (2H, s), 4.43 (2H, s),
4.50 (2H, t, J=4.4 Hz), 7.07 (2H, d, J=8.4 Hz),
7.12-7.22 (7H, m), 7.31 (1H, d, J=7.6 Hz),
7.42-7.60 (2H, m), 7.75 (1H, d, J=6.8 Hz).

### Example 15

Production of 3-{4′-{[4-butyl-6-(2-hydroxyethoxy)-2-methylpyrimidin-5-yl]methyl}-[1,1′-biphenyl]-2-yl}-1,2,4-oxadiazol-5(4H)-one

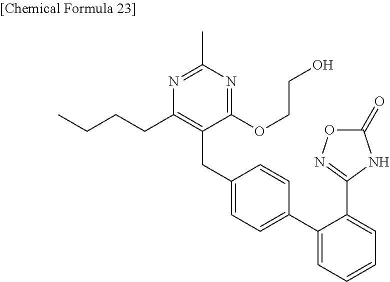

Process 1:

3-{4′-{{4-butyl-6-{2-[(tert-butyldimethylsilyl)oxy]ethoxy}-2-methylpyrimidin-5-yl}methyl}-[1,1′-biphenyl]-2-yl}-1,2,4-oxadiazol-5(4H)-one was obtained as a pale yellow oil (yield 63%) according to the same reaction and treatment as the Processes 2 and 3 of the Example 1 by using 4′-{{4-butyl-6-{2-[(tert-butyldimethylsilyl)oxy]ethoxy}-2-methylpyrimidin-5-yl}methyl}-1-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.05 (6H, s), 0.80-0.86 (12H, m), 1.21-1.32 (2H, m),
1.38-1.52 (2H, m), 2.42 (3H, s), 2.48-2.55 (2H, m),
3.86 (2H, t, J=5 Hz), 3.89 (2H, s), 4.40 (2H, t, J=5 Hz),
7.06 (2H, d, J=8 Hz), 7.11 (2H, d, J=8 Hz),
7.24-7.46 (3H, m), 7.57 (1H, d, J=8 Hz).

Process 2: Tetrabutylammonium fluoride (1 M tetrahydrofuran solution, 1.1 mL) was added to a tetrahydrofuran (2 mL) solution of 3-{4′-{{4-butyl-6-{2-[(tert-butyldimethylsilyl)oxy]ethoxy}-2-methylpyrimidin-5-yl}methyl}-[1,1′-biphenyl]-2-yl}-1,2,4-oxadiazol-5(4H)-one (120 mg, 0.2 mmol) and stirred for 6 hours at room temperature. The reaction mixture was added water and extracted with ethyl acetate. The organic layer was combined, washed with water and brine, dried over anhydrous sodium sulfate, and concentrated in vacuo. The residues obtained were subjected to preparative thin layer chromatography (hexane/ethyl acetate=3:1) to obtain 3-{4′-{[4-butyl-6-(2-hydroxyethoxy)-2-methylpyrimidin-5-yl]methyl}-[1,1′-biphenyl]-2-yl}-1,2,4-oxadiazol-5(4H)-one (51 mg, 60%) as a pale yellow oil.

¹H-NMR (CDCl₃) δ:
0.92 (3H, t, J=7.2 Hz), 1.34-1.47 (2H, m),
1.59-1.69 (2H, m), 2.55 (3H, s), 2.73-2.81 (2H, m),
3.64-3.71 (2H, m), 3.98 (2H, s), 4.32 (2H, t, J=4.4 Hz),
7.13 (2H, d, J=8.2 Hz), 7.25 (2H, d, J=8.2 Hz),
7.38-7.46 (2H, m), 7.56 (1H, t, J=7.0 Hz),
7.74 (1H, d, J=8.0 Hz).

### Example 16

Production of 3-{4′-{[4-butyl-6-(2-isopropoxyethoxy)-2-methylpyrimidin-5-yl]methyl}-[1,1′-biphenyl]-2-yl}-1,2,4-oxadiazol-5(4H)-one

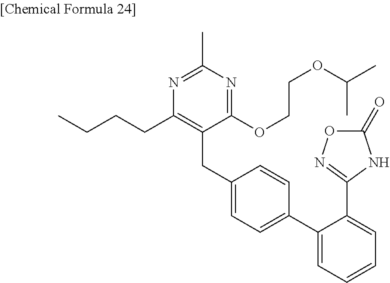

Process 1:

4′-{[4-butyl-6-(2-isopropoxyethoxy)-2-methylpyrimidin-5-yl]methyl}-1,1′-biphenyl]-2-carbonitrile was obtained as a pale yellow oil (yield 37%) according to the same reaction and treatment as the Process 1 of the Example 1 by using 4-methyl benzene 2-isopropoxyethyl sulfonate instead of the ethyl bromoacetate.

¹H-NMR (CDCl₃) δ:
0.89 (3H, t, J=7 Hz), 1.14-1.42 (8H, m), 1.51-1.61 (2H, m),
2.56 (3H, s), 2.68-2.75 (2H, m), 3.56-3.66 (1H, m),
3.72 (2H, t, J=5 Hz), 4.02 (2H, s), 4.50 (2H, t, J=5 Hz),
7.27-7.49 (6H, m), 7.58-7.65 (1H, m), 7.75 (1H, d, J=8 Hz).

Process 2:

3-{4′-{{4-butyl-6-(2-isopropoxyethoxy)-2-methylpyrimidin-5-yl}methyl}-[1,1′-biphenyl]-2-yl}-1,2,4-oxadiazol-5(4H)-one was obtained as a yellow oil (two-step yield; 44%) according to the same reaction and treatment as the Processes 2 and 3 of the Example 1 by using 4′-{[4-butyl-6-(2-isopropoxyethoxy)-2-methylpyrimidin-5-yl]methyl}-1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.88 (3H, t, J=7.2 Hz), 1.08 (6H, d, J=6.1 Hz),
1.29-1.39 (2H, m), 1.49-1.58 (2H, m), 2.43 (3H, s),
2.54-2.63 (2H, m), 3.53-3.62 (1H, m), 3.68 (2H, t, J=4.8 Hz),
3.95 (2H, s), 4.45 (2H, t, J=4.8 Hz), 7.17 (4H, brs),
7.32-7.49 (2H, m), 7.50-7.59 (1H, m), 7.73 (1H, d, J=7.1 Hz).

### Example 17

Production of 3-{4′-{{4-butyl-2-methyl-6-[2-(methylthio)ethoxy]pyrimidin-5-yl}methyl}-[1,1′-biphenyl]-2-yl}-1,2,4-oxadiazol-5(4H)-one

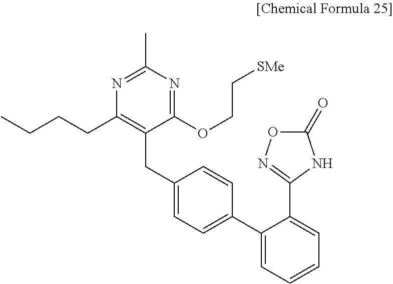

Process 1:

4′-{{4-butyl-2-methyl-6-[2-(methylthio)ethoxy]pyrimidin-5-yl}methyl}-[1,1-biphenyl]-2-carbonitrile was obtained as a pale yellow oil (yield 34%) according to the same reaction and treatment as the Process 1 of the Example 1 by using 1-chloro-2-methylthioethane instead of the ethyl bromoacetate.

¹H-NMR (CDCl₃) δ:
0.89 (3H, t, J=7 Hz), 1.28-1.41 (2H, m), 1.51-1.62 (2H, m),
2.15 (3H, s), 2.57 (3H, s), 2.66-2.74 (2H, m),
2.81 (2H, t, J=7 Hz), 4.02 (2H, s), 4.55 (2H, t, J=7 Hz),
7.26 (2H, d, J=8 Hz), 7.38-7.51 (4H, m), 7.59-7.66 (1H, m),
7.74 (1H, d, J=8 Hz).

Process 2:

3-{4′-{{4-butyl-2-methyl-6-[2-(methylthio)ethoxy]pyrimidin-5-yl}methyl}-[1,1′-biphenyl]-2-yl}-1,2,4-oxadiazol-5(4H)-one was obtained as a pale yellow oil (two-step yield; 59%) according to the same reaction and treatment as the Processes 2 and 3 of the Example 1 by using 4′-{{4-butyl-2-methyl-6-[2-(methylthio)ethoxy]pyrimidin-5-yl}methyl}-[1,1-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.90 (3H, t, J=7.2 Hz), 1.32-1.42 (2H, m),
1.52-1.62 (2H, m), 2.12 (3H, s), 2.48 (3H, s),
2.57-2.65 (2H, m), 2.78 (2H, t, J=6.8 Hz), 3.98 (2H, s),
4.53 (2H, t, J=6.8 Hz), 7.22 (4H, brs), 7.38-7.63 (3H, m),
7.85 (1H, dd, J=0.8, 8.0 Hz).

### Example 18

Production of 3-{4′-{{4-butyl-2-methyl-6-[2-(methylsulfonyl)ethoxy]pyrimidin-5-yl}methyl}-[1,1′-biphenyl]-2-yl}-1,2,4-oxadiazol-5(4H-one

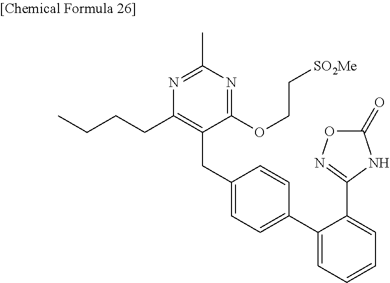

30% hydrogen peroxide (17 mg, 0.15 mmol) was added to a methanol (1.5 mL) solution of 3-{4′-{{4-butyl-2-methyl-6-[2-(methylthio)ethoxy]pyrimidin-5-yl}methyl}-[1,1′-biphenyl]-2-yl}-1,2,4-oxadiazol-5(4H)-one (14 mg, 0.030 mmol) and tantalum pentachloride (1 mg, 0.0030 mmol) and stirred overnight at room temperature. The reaction mixture was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=1:1) to obtain 3-{4′-{{4-butyl-2-methyl-6-[2-(methylsulfonyl)ethoxy]pyrimidin-5-yl}methyl}-[1,1′-biphenyl]-2-yl}-1,2,4-oxadiazol-5(4H)-one (13 mg, 86%) as a pale yellow solid.

¹H-NMR (CDCl₃) δ:
0.91 (3H, t, J=7.2 Hz), 1.34-1.45 (2H, m),
1.56-1.68 (2H, m), 2.57 (3H, s), 2.71-2.77 (2H, m),
2.84 (3H, s), 3.37 (2H, t, J=5.6 Hz), 4.00 (2H, s),
4.82 (2H, t, J=5.6 Hz), 7.12 (2H, d, J=8.2 Hz),
7.24 (2H, d, J=8.2 Hz), 7.41-7.64 (3H, m),
7.82 (1H, dd, J=1.0, 7.8 Hz).

### Example 19

Production of 3-{4′-{[4-butyl-6-(2,2-dimethoxyethoxy)-2-methylpyrimidin-5 -yl]methyl}-[1,1′-biphenyl]-2-yl}-1,2,4-oxadiazol-5(4H)-one

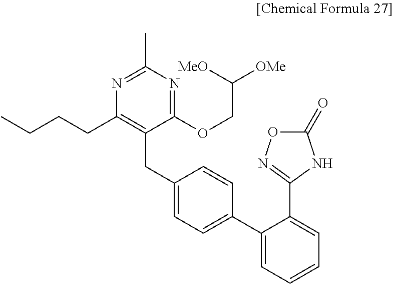

Process 1:

4′-{[4-butyl-6-(2,2-dimethoxyethoxy)-2-methylpyrimidin-5-yl]methyl}-[1,1′-biphenyl]-2-carbonitrile was obtained as a pale yellow oil (yield 10%) according to the same reaction and treatment as the Process 1 of the Example 1 by using 2-bromo-1,1-dimethoxyethane instead of the ethyl bromoacetate.

¹H-NMR (CDCl₃) δ:
0.89 (3H, t, J=7 Hz), 1.32-1.42 (2H, m), 1.52-1.62 (2H, m),
2.57 (3H, s), 2.68-2.74 (2H, m), 3.37 (6H, s),
4.02 (2H, s), 4.41 (2H, d, J=6 Hz), 4.69 (1H, t, J=6 Hz),
7.28 (2H, d, J=8.0 Hz), 7.38-7.49 (4H, m), 7.58-7.65 (1H, m),
7.74 (1H, d, J=8 Hz).

Process 2:

3-{4′-{[4-butyl-6-(2,2-dimethoxyethoxy)-2-methylpyrimidin-5-yl]methyl}-[1,1′-biphenyl]-2-yl}-1,2,4-oxadiazol-5(4H)-one was obtained as a pale yellow oil (two-step yield; 35%) according to the same reaction and treatment as the Processes 2 and 3 of the Example 1 by using 4′-{[4-butyl-6-(2,2-dimethoxyethoxy)-2-methylpyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.91 (3H, t, J=7.2 Hz), 1.32-1.44 (2H, m),
1.53-1.64 (2H, m), 2.50 (3H, s), 2.63-2.69 (2H, m),
3.28 (6H, s), 3.98 (2H, s), 4.36 (2H, d, J=5.6 Hz),
4.71 (1H, t, J=5.6 Hz), 7.18 (2H, d, J=8.3 Hz),
7.23 (2H, d, J=8.3 Hz), 7.42-7.52 (2H, m),
7.58-7.64 (1H, m), 7.82 (1H, d, J=7.8 Hz).

### Example 20

Production of 3-{4′-{[4-butyl-6-(2,2-diethoxyethoxy)-2-methylpyrimidin-5-yl]methyl}-[1,1′-biphenyl]-2-yl}-1,2,4-oxadiazol-5(4H)-one

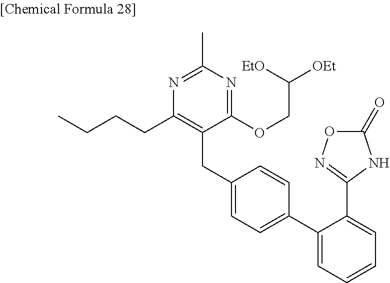

Process 1:

4′-{[4-butyl-6-(2,2-diethoxyethoxy)-2-methylpyrimidin-5-yl]methyl}-[1,1′-biphenyl]-2-carbonitrile was obtained as a pale yellow oil (yield 10%) according to the same reaction and treatment as the Process 1 of the Example 1 by using 2-bromo-1,1-diethoxyethane instead of the ethyl bromoacetate.

¹H-NMR (CDCl₃) δ:
0.89 (3H, t, J=7 Hz), 1.22 (6H, t, J=7 Hz),
1.31-1.43 (2H, m), 1.51-1.61 (2H, m), 2.57 (3H, s),
2.64-2.74 (2H, m), 3.52-3.62 (2H, m), 3.66-3.76 (2H, m),
4.01 (2H, s), 4.36-4.45 (2H, m), 4.63 (1H, t, J=5 Hz),
7.24-7.34 (2H, m), 7.38-7.51 (4H, m), 7.57-7.65 (1H, m),
7.74 (1H, d, J=8 Hz).

Process 2:

3-{4′-{[4-butyl-6-(2,2-diethoxyethoxy)-2-methylpyrimidin-5-yl]methyl}-[1,1′-biphenyl]-2-yl}-1,2,4-oxadiazol-5(4H)-one was obtained as a pale yellow oil (two-step yield; 35%) according to the same reaction and treatment as the Processes 2 and 3 of the Example 1 by using 4′-{[4-butyl-6-(2,2-diethoxyethoxy)-2-methylpyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.91 (3H, t, J=7.2 Hz), 1.12 (3H, t, J=7.1 Hz),
1.28-1.42 (5H, m), 1.50-1.63 (2H, m), 2.48 (3H, s),
2.52-2.68 (2H, m), 3.52-3.64 (4H, m), 3.98 (2H, s),
4.33-4.43 (2H, m), 4.83 (1H, t, J=5.6 Hz), 7.22 (4H, brs),
7.36-7.53 (2H, m), 7.57-7.63 (1H, m),
7.83 (1H, d, J=7.6 Hz).

### Example 21

Production of 3-{4′-{[4-butyl-6-(2-hydroxypropoxy)-2-methylpyrimidin-5-yl]methyl}-[1,1′-biphenyl]-2-yl}-1,2,4-oxadiazol-5(4H)-one

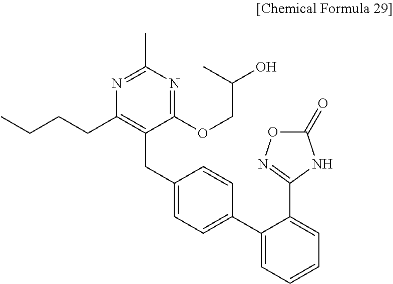

Process 1:

4′-{{4-butyl-6-{2-[(tert-butyldiphenylsilyl)oxy]propoxy}-2-methylpyrimidin-5-yl}methyl}-N′-hydroxy-[1,1′-biphenyl]-2-carboxy imidamide was obtained as a pale yellow oil (yield 18%) according to the same reaction and treatment as the Process 1 of the Example 1 by using [(1-bromopropan-2-yl)oxy](tert-butyl)diphenylsilane instead of the ethyl bromoacetate.

¹H-NMR (CDCl₃) δ:
0.89-0.95 (12H, m), 0.98 (3H, d, J=6 Hz), 1.34-1.45 (2H, m),
1.54-1.64 (2H, m), 2.55-2.69 (5H, m), 3.69-3.79 (1H, m),
3.89-3.99 (2H, m), 4.21-4.46 (2H, m), 7.24-7.66 (14H, m),
7.56-7.65 (3H, m), 7.74 (1H, d, J=8 Hz).

Process 2:

3-{4′-{[4-butyl-6-(2-[(tert-butyldiphenylsilyl)oxy]pr opoxy)-2-methylpyrimidin-5-yl]methyl}-[1,1′-biphenyl]-2-yl}-1,2,4-oxadiazol-5(4H)-one was obtained as a pale yellow oil (yield 50%) according to the same reaction and treatment as the Processes 2 and 3 of the Example 1 by using 4′-{{4-butyl-6-{2-[(tert-butyldiphenylsilyl)oxy]propoxy}-2-methylpyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.89 (3H, t, J=7 Hz), 0.96-1.01 (12H, m), 1.26-1.36 (2H, m),
1.45-1.57 (2H, m), 2.24 (3H, s), 2.22-2.39 (2H, m),
3.76 (2H, s), 4.05-4.17 (2H, m), 4.24-4.34 (1H, m),
7.03 (2H, d, J=8 Hz), 7.13 (2H, d, J=8 Hz),
7.25-7.66 (13H, m), 7.78 (1H, d, J=8 Hz).

Process 3:

3-{4′-{[4-butyl-6-(2-hydroxypropoxy)-2-methylpyrimidin-5-yl]methyl}-[1,1′-biphenyl]-2-yl}-1,2,4-oxadiazol-5(4H)-one was obtained as a pale yellow oil (two-step yield; 69%) according to the same reaction and treatment as the Process 3 of the Example 15 by using 3-{4′-{[4-butyl-6-(2-[(tert-butyldiphenylsilyl)oxy]propoxy)-2-methylpyrimidin-5-yl]methyl}-[1,1′-biphenyl]-2-yl}-1,2,4-oxadiazol-5(4H)-one instead of the 3-{4′-{{4-butyl-6-{2-[(tert-butyldimethylsilyl)oxy]ethoxy}-2-methylpyrimidin-5-yl}methyl}-[1,1′-biphenyl]-2-yl}-1,2,4-oxadiazol-5(4H)-one.

¹H-NMR (CDCl₃) δ:
0.95 (3H, t, J=7.3 Hz), 1.12 (3H, d, J=6.1 Hz),
1.39-1.51 (2H, m), 1.64-1.77 (2H, m), 2.58 (3H, s),
2.78-2.89 (2H, m), 3.86-4.37 (5H, m), 7.21 (2H, d, J=8.2 Hz),
7.35 (2H, d, J=8.2 Hz), 7.46-7.66 (3H, m),
7.81 (1H, d, J=7.8 Hz).

### Example 22

Production of 3-{4′-{[4-butyl-2-methyl-6-(2-oxopropoxy)pyrimidin-5-yl]methyl}-[1,1′-biphenyl]-2-yl}-1,2,4-oxadiazol-5(4H)-one

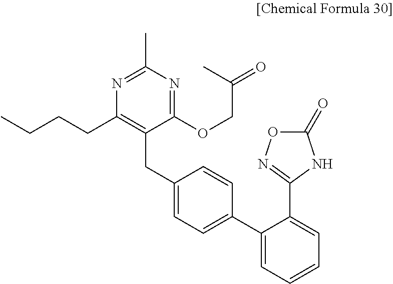

Process 1: Dess-Martin periodinane (590 mg, 1. 4 mmol) was added to a dichloromethane (5 mL) solution of 3-{4′-{[4-butyl-6-(2-hydroxypropoxy)-2-methylpyrimidin-5-yl]methyl}-[1,1′-biphenyl]-2-yl}-1,2,4-oxadiazol-5(4H)-one (330 mg, 0.70 mmol) and stirred for 2 hours at room temperature. The reaction mixture was added water and extracted with ethyl acetate. The organic layer was combined, washed with water and brine, dried over anhydrous sodium sulfate, and concentrated in vacuo. The residues obtained were subjected to silica gel column chromatography (chloroform/methanol) to obtain 3-{4′-{[4-butyl-2-methyl-6-(2-oxopropoxy)pyrimidin-5-yl]methyl}-[1,1′-biphenyl]-2-yl}-1,2,4-oxadiazol-5(4H)-one (301 mg, 95%) as a pale yellow solid.

¹H-NMR (CDCl₃) δ:
0.91 (3H, t, J=7.3 Hz), 1.33-1.43 (2H, m),
1.54-1.66 (2H, m), 2.12 (3H, s), 2.45 (3H, s),
2.63-2.72 (2H, m), 4.03 (2H, s), 4.94 (2H, s),
7.18-7.27 (4H, m), 7.38-7.53 (2H, m),
7.60 (1H, dt, J=1.4, 7.6 Hz), 7.88 (1H, d, J=7.6 Hz).

### Example 23

Production of 3-{4′-{[4-butyl-6-(4-methoxybutoxy)-2-methylpyrimidin-5-yl]methyl}-[1,1′-biphenyl]-2-yl}-1,2,4-oxadiazol-5(4H)-one

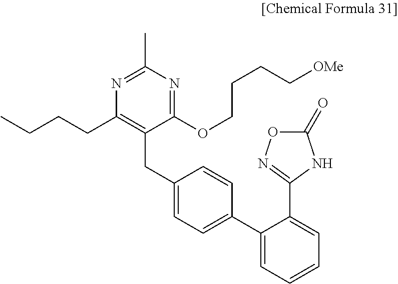

Process 1:

4′-{{4-butyl-6-{4-[(tert-butyldiphenylsilyl)oxy]butoxy}-2-methylpyrimidin-5-yl}methyl}-[1,1′-biphenyl]-2-carbonitrite was obtained as a pale yellow oil (yield 60%) according to the same reaction and treatment as the Process 1 of the Example 1 by using (4-bromobutoxy)(tert-butyl)diphenylsilane instead of the ethyl bromoacetate.

¹H-NMR (CDCl₃) δ:
0.88 (3H, t, J=7 Hz), 1.00-1.08 (9H, m), 1.32-1.41 (2H, m),
1.51-1.64 (4H, m), 1.76-1.86 (2H, m), 2.56 (3H, s),
2.65-2.74 (2H, m), 3.66 (2H, t, J=6 Hz), 3.97 (2H, s),
4.35 (2H, t, J=6 Hz), 7.19-7.76 (18H, m).

Process 2:

4′-{[4-butyl-6-(4-hydroxybutoxy)-2-methylpyrimidin-5-yl]methyl}-[1,1′-biphenyl]-2-carbonitrile was obtained as a pale yellow oil (yield 63%) according to the same reaction and treatment as the Process 2 of the Example 12 by using 4′-{{4-butyl-6-{4-[(tert-butyldiphenylsilyl)oxy]butoxy}-2-methylpyrimidin-5-yl}methyl}-[1,1′-biphenyl]-2-carbonitrile instead of the 4′-{{4-butyl-6-{2-[(tert-butyldimethylsilyl)oxy]ethoxy}-2-methylpyrimidin-5-yl}methyl}-1-[1,1′-biphenyl]-2-carbonitrile.

¹H-NMR (CDCl₃) δ:
0.90 (3H, t, J=7 Hz), 1.32-1.44 (2H, m), 1.48-1.65 (4H, m),
1.72-1.81 (2H, m), 2.58 (3H, s), 2.67-2.76 (2H, m),
3.59 (2H, t, J=6 Hz), 4.01 (2H, s), 4.37 (2H, t, J=6 Hz),
7.22 (2H, d, J=8 Hz), 7.38-7.52 (4H, m), 7.58-7.67 (1H, m),
7.74 (1H, d, J=8 Hz).

Process 3:

4′-{[4-butyl-6-(4-methoxybutoxy)-2-methylpyrimidin-5-yl]methyl}-[1,1′-biphenyl]-2-carbonitrile was obtained as a pale yellow oil (yield 60%) according to the same reaction and treatment as the Process 3 of the Example 13 by using 4′-{[4-butyl-6-(4-hydroxybutoxy)-2-methylpyrimidin-5-yl]methyl}-[1,1′-biphenyl]-2-carbonitrile instead of the N,N-dimethylformamide of the 4′-{[4-butyl-6-(2-hydroxyethoxy)-2-methylpyrimidin-5-yl]methyl}-[1,1′biphenyl]-2-carbonitrile (80 mg, 0.20 mmol) and iodomethane instead of the n-iodopropane.

¹H-NMR (CDCl₃) δ:
0.89 (3H, t, J=7 Hz), 1.32-1.43 (2H, m), 1.52-1.66 (4H, m),
1.74-1.83 (2H, m), 2.57 (3H, s), 2.66-2.75 (2H, m),
3.29 (3H, s), 3.36 (2H, t, J=6 Hz), 4.00 (2H, s),
4.36 (2H, t, J=6 Hz), 7.24 (2H, d, J=8 Hz),
7.36-7.52 (4H, m), 7.58-7.65 (1H, m), 7.74 (1H, d, J=8 Hz).

Process 4:

3-{4′-{[4-butyl-6-(4-methoxybutoxy)-2-methylpyrimidin-5-yl]methyl}-[1,1′-biphenyl]-2-yl}-1,2,4-oxadiazol-5(4H)-one was obtained as a pale yellow oil (two-step yield; 39%) according to the same reaction and treatment as the Processes 2 and 3 of the Example 1 by using 4′-{[4-butyl-6-(4-methoxybutoxy)-2-methylpyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.92 (3H, t, J=7.2 Hz), 1.10-1.21 (2H, m),
1.36-1.71 (6H, m), 2.58 (3H, s), 2.69-2.78 (2H, m),
2.90 (3H, s), 3.14 (2H, t, J=8.0 Hz), 3.97 (2H, s),
4.24 (2H, t, J=5.8 Hz), 7.09 (2H, d, J=8.3 Hz),
7.28 (2H, d, J=8.3 Hz), 7.44-7.53 (2H, m),
7.58-7.65 (1H, m), 7.69 (1H, d, J=7.6 Hz).

### Example 24

Production of 3-{4′-{{4-[(1,3-dioxolan-2-yl)methoxy]-6-butyl-2-methylpyrimidin-5-yl}methyl}-[1,1′-biphenyl]-2-yl}-1,2,4-oxadiazol-5(4H)-one

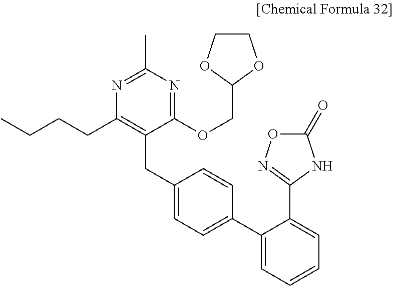

Process 1:

4′-{{4-[(1,3-dioxolan-2-yl)methoxy]-6-butyl-2-methylpyrimidin-5-yl}methyl}-[1,1′-biphenyl]-2-carbonitrile was obtained as a pale yellow oil (yield 12%) according to the same reaction and treatment as the Process 1 of the Example 1 by using 2-(bromomethyl)-1,3-dioxolane instead of the ethyl bromoacetate.

¹H-NMR (CDCl₃) δ:
0.89 (3H, t, J=7 Hz), 1.31-1.41 (2H, m), 1.50-1.61 (2H, m),
2.57 (3H, s), 2.66-2.74 (2H, m), 3.85-3.99 (4H, m),
4.04 (2H, s), 4.46 (2H, d, J=4 Hz), 5.26 (1H, t, J=4 Hz),
7.28 (2H, d, J=8 Hz), 7.38-7.51 (4H, m),
7.58-7.65 (1H, m), 7.74 (1H, d, J=8 Hz).

Process 2:

3-{4′-{{4-[(1,3-dioxolan-2-yl)methoxy]-6-butyl-2-methylpyrimidin-5-yl}methyl}-[1,1′-biphenyl]-2-yl}-1,2,4-oxadiazol-5 (4H)-one was obtained as a pale yellow oil (two-step yield; 53%) according to the same reaction and treatment as the Processes 2 and 3 of the Example 1 by using 4′-{{4-[(1,3-dioxolan-2-yl)methoxy]-6-butyl-2-methylpyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.88 (3H, t, J=7.2 Hz), 1.30-1.41 (2H, m),
1.51-1.61 (2H, m), 2.51 (3H, s), 2.61-2.68 (2H, m),
3.82-3.93 (4H, m), 3.97 (2H, s), 4.38 (2H, d, J=5.6 Hz),
5.21 (1H, t, J=5.6 Hz), 7.14 (2H, d, J=8.3 Hz),
7.17 (2H, d, J=8.3 Hz), 7.33-7.45 (2H, m),
7.50-7.58 (1H, m), 7.73 (1H, d, J=7.6 Hz).

### Example 25

Production of methyl 2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetate

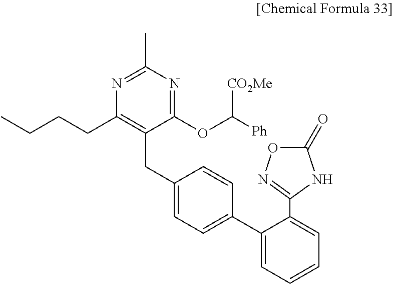

Process 1: Methyl 2-{{6-butyl-5-[(2′-cyano-[1,1′-biphenyl]-4-yl)methyl]-2-methylpyrimidin-4-yl}oxy}-2-phenylacetate was obtained as a pale yellow oil (yield 35%) according to the same reaction and treatment as the Process 1 of the Example 1 by using methyl 2-bromo-2-phenylacetate instead of the ethyl bromoacetate.

¹H-NMR (CDCl₃) δ:
0.88 (3H, t, J=7 Hz), 1.31-1.42 (2H, m), 1.47-1.61 (2H, m),
2.56 (3H, s), 2.66-2.74 (2H, m), 3.72 (3H, s),
4.02-4.20 (2H, m), 6.25 (1H, s), 7.33-7.51 (11H, m),
7.58-7.64 (1H, m), 7.74 (1H, d, J=8 Hz).

Process 2: Methyl 2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetate was obtained as a pale yellow oil (two-step yield; 38%) according to the same reaction and treatment as the

Processes 2 and 3 of the Example 1 by using methyl 2-{{6-butyl-5-[(2′-cyano-[1,1′-biphenyl]-4-yl)methyl]-2-methylpyrimidin-4-yl}oxy}-2-phenylacetate instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.90 (3H, t, J=7.2 Hz), 1.33-1.43 (2H, m),
1.51-1.63 (2H, m), 2.53 (3H, s), 2.68-2.76 (2H, m),
3.69 (3H, s), 4.00-4.14 (2H, m), 6.31 (1H, s),
7.20-7.64 (12H, m), 7.85 (1H, d, J=7.8 Hz).

### Example 26

Production of 2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetic acid

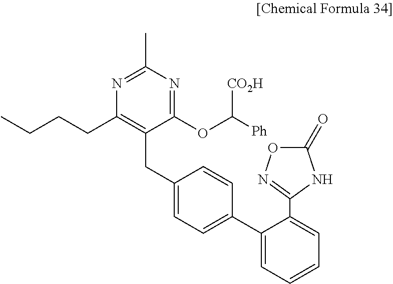

2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetic acid was obtained as a pale yellow solid (yield 82%) according to the same reaction and treatment as the Process 1 of the Example 2 by using methyl 2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetate instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.92 (3H, t, J=7.2 Hz), 1.36-1.51 (2H, m),
1.64-1.79 (2H, m), 2.72 (3H, s), 2.91-3.18 (2H, m),
3.97-4.16 (2H, m), 6.65 (1H, s), 7.19-7.64 (12H, m),
7.99 (1H, d, J=7.8 Hz).

### Example 27

Production of methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionate

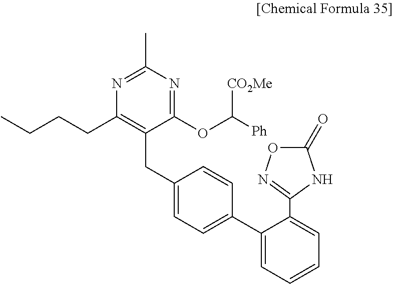

Process 1: Methyl 2-{{6-butyl-5-[(2′-cyano-[1,1′-biphenyl]-4-yl)methyl]-2-methylpyrimidin-4-yl}oxy}-3-phenylpropionate was obtained as a pale yellow oil (yield 59%) according to the same reaction and treatment as the Process 1 of the Example 1 by using methyl 2-bromo-3-phenylpropionate instead of the ethyl bromoacetate.

¹H-NMR (CDCl₃) δ:
0.85 (3H, t, J=7 Hz), 1.27-1.37 (2H, m), 1.43-1.52 (2H, m),
2.48 (3H, s), 2.58-2.67 (2H, m), 3.12-3.28 (2H, m),
3.71 (3H, s), 3.96-4.08 (2H, m), 5.48-5.54 (1H, m),
7.17-7.49 (11H, m), 7.58-7.66 (1H, m), 7.81 (1H, d, J=8 Hz).

Process 2: Methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionate was obtained as a pale yellow oil (two-step yield; 24%) according to the same reaction and treatment as the Process 2 of the Example 1 by using methyl 2-{{6-butyl-5-[(2′-cyano-[1,1′-biphenyl]-4-yl)methyl]-2-methylpyrimidin-4-yl}oxy}-3-phenylpropionate instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:

0.88 (3H, t, J=7.2 Hz), 1.27-1.39 (2H, m), 1.45-1.58 (2H, m), 2.42 (3H, s), 2.55-2.66 (2H, m), 3.12-3.38 (2H, m), 3.69 (3H, s), 3.90-4.03 (2H, m), 5.52-5.58 (1H, m), 7.11-7.52 (12H, m), 7.81 (1H, brs).

### Example 28

Production of 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionic acid

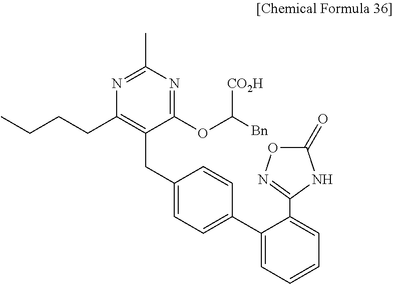

Process 1:

2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionoic acid was obtained as a pale yellow oil (yield 44%) according to the same reaction and treatment as the Process 1 of the Example 2 by using methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionate instead of the ethyl 2-{{6-butyl-5-[(2′-cyano-[1,1′-biphenyl]-4-yl)methyl]-2-methylpyrimidin-4-yl}oxy}acetate.

¹H-NMR (CD₃OD ) δ:
0.86 (3H, t, J=7.2 Hz), 1.27-1.43 (4H, m), 2.51 (3H, s),
2.63-2.69 (2H, m), 3.18-3.38 (2H, m), 4.00-4.09 (2H, m),
5.62-5.67 (1H, m), 7.10-7.68 (13H, m).

### Example 29

Production of 3-{4′-{{4-butyl-2-methyl-6-[2-oxo-2-(pyrrolidin-1-yl)ethoxy]pyrimidin-5-yl}methyl}-[1,1′-biphenyl]-2-yl}-1,2,4-oxadiazof-5 (4H)-one

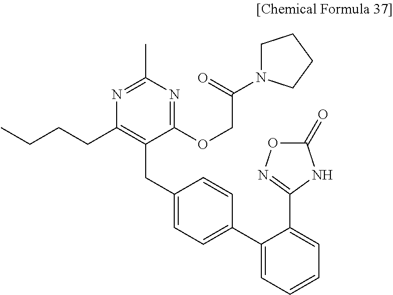

Process 1:

4′-{{4-butyl-2-methyl-6-[2-oxo-2- (pyrrolidin-1-yl)ethoxy]pyrimidin-5-yl}methyl}-[1,1′-biphenyl]-2-carbonitrile was obtained as a pale yellow amorphous (yield 100%) according to the same reaction and treatment as the Process 1 of the Example 3 by using pyrrole instead of the ammonia.

¹H-NMR (CDCl₃) δ:
0.87 (3H, t, J=7 Hz), 1.29-1.41 (2H, m), 1.48-1.58 (2H, m),
1.81-2.02 (4H, m), 2.54 (3H, s), 2.64-2.72 (2H, m),
3.46 (2H, t, J=7 Hz), 3.51 (2H, t, J=7 Hz), 4.11 (2H, s),
4.95 (2H, s), 7.33 (2H, d, J=8 Hz), 7.38-7.50 (4H, m),
7.58-7.64 (1H, m), 7.74 (1H, d, J=8 Hz).

Process 2:

3-{4′-{{4-butyl-2-methyl-6-[2-oxo-2-(pyrrolidin-1-yl)ethoxy]pyrimidin-5-yl}methyl}-[1,1′-biphenyl]-2-yl}-1,2,4-oxadiazol-5 (4H)-one was obtained as a pale yellow oil (two-step yield; 50%) according to the same reaction and treatment as the Processes 2 and 3 of the Example 1 by using 4′-{{4-butyl-2-methyl-6-[2-oxo-2-(pyrrolidin-1-yl)ethoxy]pyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.94 (3H, t, J=7.2 Hz), 1.36-1.49 (2H, m),
1.61-1.82 (4H, m), 1.93-1.99 (2H, m), 2.49 (3H, s),
2.74-2.84 (2H, m), 3.28 (2H, t, J=6.8 Hz),
3.52 (2H, t, J=6.8 Hz), 4.00 (2H, s), 4.84 (2H, s),
7.16 (2H, d, J=8.3 Hz), 7.22 (2H, d, J=8.3 Hz),
7.43-7.63 (3H, m), 7.78 (1H, d, J=7.8 Hz).

### Example 30

Production of 3-{4′-{{4-butyl-2-methyl-6-[2-oxo-2-(piperidin-1-yflethoxy]pyrimidin-5-yl}methyl}-[1,1′-biphenyl]-2-yl}-1,2,4-oxadiazol-5 (4H)-one

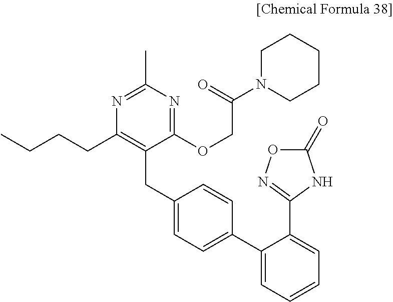

Process 1:

4′-{{4-butyl-2-methyl-6-[2-oxo-2-(piperidin-1-yflethoxy]pyrimidin-5-yl}methyl}-[1,1′-biphenyl]-2-carbonitrile was obtained as a pale yellow amorphous (yield 100%) according to the same reaction and treatment as the Process 1 of the Example 3 by using piperidine instead of the ammonia.

¹H-NMR (CDCl₃) δ:
0.87 (3H, t, J=7 Hz), 1.28-1.39 (2H, m), 1.48-1.70 (8H, m),
2.55 (3H, s), 2.64-2.72 (2H, m), 3.32-3.38 (2H, m),
3.52-3.59 (2H, m), 4.10 (2H, s), 5.06 (2H, s),
7.33 (2H, d, J=8 Hz), 7.38-7.49 (4H, m), 7.56-7.64 (1H, m),
7.73 (1H, d, J=8 Hz).

Process 2:

3-{4′-{{4-butyl-2-methyl-6-[2-oxo-2-(piperidin-1-yl)ethoxy]pyrimidin-5-yl}methyl}-[1,1′-biphenyl]-2-yl}-1,2,4-oxadiazol-5(4H)-one was obtained as a pale yellow oil (two-step yield; 57%) according to the same reaction and treatment as the Processes 2 and 3 of the Example 1 by using 4′-{{4-butyl-2-methyl-6-[2-oxo-2-(piperidin-1-yl}ethoxy]pyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.93 (3H, t, J=7.2 Hz), 1.36-1.53 (4H, m),
1.58-1.72 (6H, m), 2.50 (3H, s), 2.74-2.84 (2H, m),
3.34-3.43 (4H, m), 4.01 (2H, s), 4.93 (2H, s),
7.18 (2H, d, J=8.4 Hz), 7.22 (2H, d, J=8.4 Hz),
7.43-7.64 (3H, m), 7.79 (1H, d, J=7.6 Hz).

### Example 31

Production of 3-{4′-{[4-butyl-2-methyl-6-(2-morpholino-2-oxoethoxy)pyrimidin-5-yl]methyl}-[1,1′-biphenyl]-2-yl}-1,2,4-oxadiazol-5(4H-one

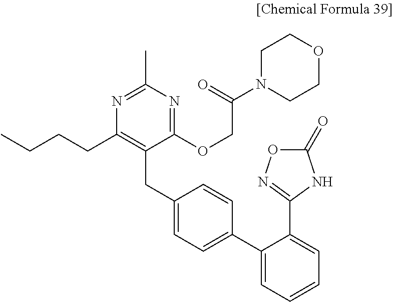

Process 1:

4′-{[4-butyl-2-methyl-6-(2-morpholino-2-oxoethoxy)pyrimidin-5-yl]methyl}-[1,1′-biphenyl]-2-carbonitrile was obtained as a pale yellow amorphous (yield 100%) according to the same reaction and treatment as the Process 1 of the Example 3 by using morpholine instead of the ammonia.

¹H-NMR (CDCl₃) δ:
0.87 (3H, t, J=7 Hz), 1.30-1.41 (2H, m), 1.49-1.61 (2H, m),
2.56 (3H, s), 2.66-2.73 (2H, m), 3.38-3.72 (8H, m),
4.10 (2H, s), 5.04 (2H, s), 7.31 (2H, d, J=8 Hz),
7.38-7.51 (4H, m), 7.57-7.66 (1H, m), 7.74 (1H, d, J=8 Hz).

Process 2:

3-{4′-{[4-butyl-2-methyl-6-(2-morpholino-2-oxoethoxy)pyrimidin-5-yl]methyl}-[1,1′-biphenyl]-2-yl}-1,2,4-oxadiazol-5 (4H)-one was obtained as a pale yellow oil (two-step yield; 55%) according to the same reaction and treatment as the Processes 2 and 3 of the Example 1 by using the 4′-{[4-butyl-2-methyl-6-(2-morpholino-2-oxoethoxy)pyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.93 (3H, t, J=7.2 Hz), 1.36-1.48 (2H, m),
1.61-1.72 (2H, m), 2.51 (3H, s), 2.75-2.83 (2H, m),
3.42-3.50 (4H, m), 3.58-3.73 (4H, m), 4.02 (2H, s),
4.94 (2H, s), 7.18 (2H, d, J=8.0 Hz),
7.23 (2H, d, J=8.0 Hz), 7.43-7.64 (3H, m),
7.79 (1H, d, J=7.6 Hz).

### Example 32

Production of 3-{4′-{{4-butyl-2-methyl-6-{[5-methyl-2-(p-tolyl)oxazol-4-yl]methoxy}pyrimidin-5-yl}methyl}-[1,1′-biphenyl]-2-yl}-1,2,4-oxadiazol-5(4H)-one

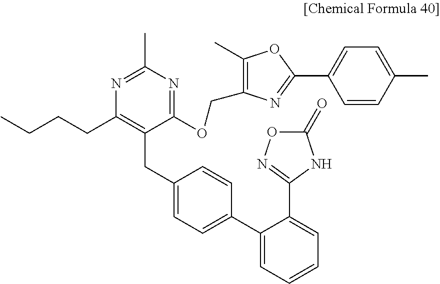

Process 1:

4′-{{4-butyl-2-methyl-6-{[5-methyl-2-(p-tolyl)oxazol-4-yl]methoxy}pyrimidin-5-yl}methyl}-[1,1′-biphenyl]-2-carbonitrile was obtained as a white amorphous according to the same reaction and treatment as the the Process 1 of the Example 1 by using 4-(bromomethyl)-5-methyl-2-(p-tolyl)oxazole instead of the ethyl bromoacetate.

¹H-NMR (CDCl₃) δ:
0.88 (3H, t, J=7 Hz), 1.30-1.40 (2H, m), 1.50-1.58 (2H, m),
2.44 (3H, s), 2.61 (3H, s), 2.69 (2H, t, J=8 Hz),
4.02 (2H, s), 5.38 (2H, s), 7.23-7.26 (2H, m),
7.37-7.43 (7H, m), 7.56 (1H, td, J=8 Hz, 1 Hz),
7.72 (1H, d, J=8 Hz), 7.97-8.01 (2H, m).

Process 2:

3-{4′-{{4-butyl-2-methyl-6-{[5-methyl-2-(p-tolyl)oxazof-4-yl]methoxy}pyrimidin-5-yl}methyl}-[1,1′-biphenyl]-2-yl}-1,2,4-oxadiazol-5(4H)-one was obtained as a pale yellow viscous oil according to the same reaction and treatment as the Processes 2 and 3 of the Example 1 by using 4′-{{4-butyl-2-methyl-6-{[5-methyl-2-(p-tolyl)oxazol-4-yl]methoxy}pyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.94 (3H, t, J=7 Hz), 1.39-1.48 (2H, m), 1.64-1.72 (2H, m),
2.50 (3H, s), 2.58 (3H, s), 2.81 (2H, t, J=8 Hz),
3.95 (2H, s), 5.20 (2H, s), 6.81 (2H, t, J=8 Hz),
7.06 (2H, d, J=8 Hz), 7.17-7.21 (3H, m), 7.32 (2H, d, J=8 Hz),
7.53 (1H, d, J=8 Hz), 7.58 (1H, t, J=8 Hz),
7.70 (1H, t, J=7 Hz), 7.84 (1H, d, J=8 Hz).

### Example 33

Production of 3-{4′-{[4-butyl-6-(2-methoxyethoxy)-2-methylpyrimidin-5-yl]methyl}-[1,1′-biphenyl]-2-yl}-1,2,4-oxadiazol-5(4H)-one

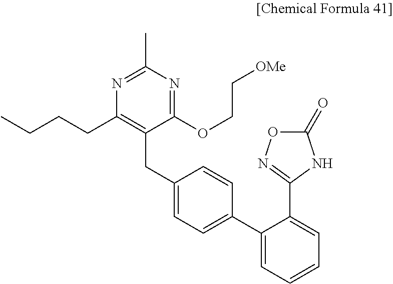

Process 1:

4′-[(4-Butyl-2-methyl-6-oxo-1,6-dihydropyrimidin-5-yl)methyl]-[1,1′-biphenyl]-2-carbonitrile (1.07 g, 2.99 mmol), 2-methoxyethanol (340 mg, 4.47 mmol), and triphenylphosphine (790 mg, 3.01 mmol) were dried for 3 hours in vacuo, replaced with argon, added tetrahydrofuran (30 mL) and diethyl azodicarboxylic acid (2.2 mol/L toluene solution, 1.4 mL, 3.08 mmol), and stirred for 5 hours at room temperature. The reaction mixture was added water and extracted with ethyl acetate. The organic layer was combined, washed with water and brine, dried over anhydrous sodium sulfate, and concentrated in vacuo. The residues obtained were subjected to silica gel column chromatography (hexane/ethyl acetate=2:1) to obtain 4′-{[4-butyl-6-(2-methoxyethoxy)-2-methylpyrimidin-5-yl]methyl}-[1,1′-biphenyl]-2-carbonitrile (683 mg, 55%) as yellow oil.

¹H-NMR (CDCl₃) δ:
0.89 (3H, t, J=7 Hz), 1.29-1.38 (2H, m), 1.52-1.63 (2H, m),
2.57 (3H, s), 2.70 (2H, t, J=7 Hz), 3.37 (3H, s),
3.69 (2H, t, J=5 Hz), 4.03 (2H, s), 4.53 (2H, t, J=5 Hz),
7.28 (2H, d, J=8 Hz), 7.38-7.51 (4H, m),
7.62 (1H, t, J=8 Hz), 7.75 (1H, d, J=8 Hz).

Process 2:

3-{4′-{[4-butyl-6-(2-methoxyethoxy)-2-methylpyrimidin-5-yl]methyl}-[1,1′-biphenyl]-2-yl}-1,2,4-oxadiazol-5 (4H)-one was obtained as a colorless oil according to the same reaction and treatment as the Processes 2 and 3 of the Example 1 by using 4′-{[4-butyl-6-(2-methoxyethoxy)-2-methylpyrimidin-5-yl]methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.92 (3H, t, J=7 Hz), 1.33-1.42 (2H, m), 1.52-1.63 (2H, m),
2.52 (3H, s), 2.72 (2H, t, J=7 Hz), 3.19 (3H, s),
3.63 (2H, t, J=5 Hz), 3.99 (2H, s), 4.45 (2H, t, J=5 Hz),
7.14-7.30 (4H, m), 7.40-7.54 (2H, m), 7.62 (1H, t, J=8 Hz),
7.85 (1H, d, J=8 Hz).

### Example 34

Production of 3-{4′-{{4-butyl-6-[(4-hydroxycyclohexyl)oxy]-2-methylpyrimidin-5-yl]methyl}-[1,1′-biphenyl]-2-yl}-1,2,4-oxadiazol-5 (4H)-one

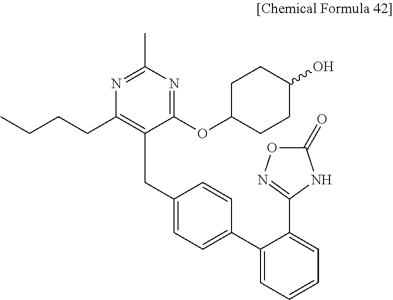

Process 1:

4′-{{4-butyl-6-{{4-[(tert-butyldimethylsilyl)oxy]cyclohexyl}oxyl-2-methylpyrimidin-5-yl}methyl}-[1,1′-biphenyl]-2-carbonitrile was obtained as a pale yellow oil (yield 28%) according to the same reaction and treatment as the Process 1 of the Example 33 by using 4-[(tert-butyldimethylsilyl)oxy]cyclohexanol instead of the 2-methoxyethanol.

¹H-NMR (CDCl₃) δ:
0.03 (6H, s), 0.82-0.93 (12H, m), 1.32-1.65 (10H, m),
1.81-1.93 (2H, m), 2.53 (3H, s), 2.67-2.73 (2H, m),
3.68-3.75 (1H, m), 3.99 (2H, s), 5.14-5.26 (1H, m),
7.20-7.29 (2H, m), 7.36-7.49 (4H, m), 7.56-7.63 (1H, m),
7.68-7.74 (1H, m).

Process 2:

3-{4′-{{4-butyl-6-{{4-[(tert-butyldimethylsilyl)oxy]cyclohexyl}oxyl-2-methylpyrimidin-5-yl}methyl}-[1,1′-biphenyl]-2-yl}-1,2,4-oxadiazol-5(4H)-one was obtained as a pale yellow oil (yield 48%) according to the same reaction and treatment as the Processes 2 and 3 of the Example 1 by using 4′-{{4-butyl-6-{{4-[(tert-butyldimethylsilyl)oxy]cyclohexyl}oxy}-2-methylpyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.05 (6H, s), 0.82-0.98 (12H, m), 1.32-1.77 (10H, m),
1.92-2.08 (2H, m), 2.40 (3H, s), 2.52-2.62 (2H, m),
3.66-3.77 (1H, m), 4.00 (2H, s), 5.11-5.18 (1H, m),
7.16-7.34 (4H, m), 7.41-7.57 (2H, m), 7.62-7.69 (1H, m),
7.76-7.84 (1H, m).

Process 3:

3-{4′-{{4-butyl-6-[(4-hydroxycyclohexyl)oxy]-2-methylpyrimidin-5-yl}methyl}-[1,1′-biphenyl]-2-yl}-1,2,4-oxadiazol-5(4H)-one was obtained as a pale yellow oil (yield 100%) according to the same reaction and treatment as the Process 3 of the Example 15 by using 3-{4′-{{4-butyl-6-{{4-[(tert-butyldimethylsilyl)oxy]cyclohexyl}oxy}-2-methylpyrimidin-5-yl}methyl}-[1,1′-biphenyl]-2-yl-1,2,4-oxadiazol-5(4H)-one instead of the 3-{4′-{{4-butyl-6-{2-[(tert-butyldimethylsilyl)oxy]ethoxy}-2-methylpyrimidin-5-yl}methyl}-[1,1′-biphenyl]-2-yl}-1,2,4-oxadiazol-5(4H)-one.

¹H-NMR (CDCl₃) δ:
0.90 (3H, t, J=7.3 Hz), 1.31-1.69 (10H, m),
1.78-2.04 (2H, m), 2.53 (3H, s), 2.58-2.64 (2H, m),
3.52-3.73 (1H, m), 4.00 (2H, s), 5.09 (1H, brs),
7.08-7.22 (2H, m), 7.27-7.38 (2H, m),
7.41-7.50 (2H, m), 7.54-7.74 (2H, m).

### Example 35

Production of 3-{4′-{{4-butyl-2-methyl-6-[(4-oxocyclohexyl)oxy]pyrimidin-5-yl}methyl}-[1,1′-biphenyl]-2-yl}-1,2,4-oxadiazol-5(4H)-one

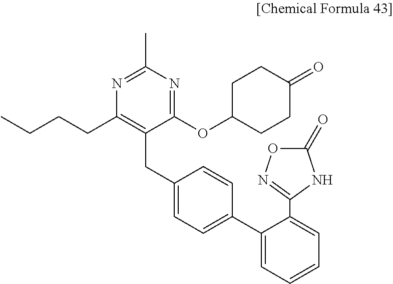

3-{4′-{{4-butyl-2-methyl-6-[(4-oxocyclohexyl)oxy]pyrimidin-5-yl}methyl}-[1,1′-biphenyl]-2-yl}-1,2,4-oxadiazol-5(4H)-one was obtained as a pale yellow oil (yield 79%) according to the same reaction and treatment as the Process 1 of the Example 22 by using 3-{4′-{{4-butyl-6-[(4-hydroxycyclohexyl)oxy]-2-methylpyrimidin-5-yl}methyl}-[1,1′-biphenyl]-2-yl}-1,2,4-oxadiazol-5(4H)-one instead of the 3-{4′-{[4-butyl-6-(2-hydroxypropoxy)-2-methylpyrimidin-5-yl]methyl}-[1,1′-biphenyl]-2-yl}-1,2,4-oxadiazol-5(4H)-one.

¹H-NMR (CDCl₃) δ:
0.92 (3H, t, J=7.3 Hz), 1.33-1.46 (2H, m),
1.58-1.71 (2H, m), 1.85-2.07 (4H, m), 2.26-2.41 (4H, m),
2.56 (3H, s), 2.68-2.78 (2H, m), 3.99 (2H, s),
5.38 (1H, brs), 7.10 (2H, d, J=8.3 Hz),
7.25 (2H, d, J=8.3 Hz), 7.38-7.51 (2H, m),
7.60 (1H, dt, J=1.4, 7.6 Hz), 7.73 (1H, d, J=7.6 Hz).

### Example 36

Production of 3-{4′-{{4-butyl-6-[3-methoxy-4-(2-methoxyethoxy)phenethoxy]-2-methylpyrimidin-5-yl}methyl}-[1,1′-biphenyl]-2-yl}-1,2,4-oxadiazol-5 (4H)-one

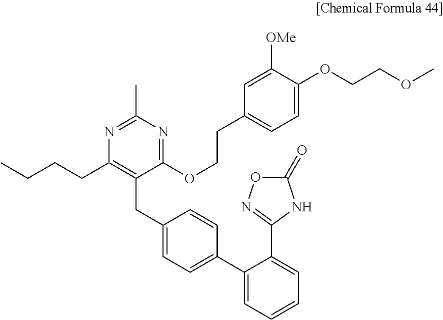

Process 1:

4′-{{4-butyl-6-[3-methoxy-4-(2-methoxyethoxy)phenethoxy]-2-methylpyrimidin-5-yl}methyl}-[1,1′-biphenyl]-2-carbonitrile was obtained as a brown oil according to the same reaction and treatment as the Process 1 of the Example 33 according to the same reaction and treatment as the Process 1 of the Example 33 by using 2-[3-methoxy-4-(2-methoxyethoxy)phenyl]ethanol instead of the 2-methoxyethanol.

¹H-NMR (CDCl₃) δ:
0.88 (3H, t, J=8 Hz), 1.33-1.42 (2H, m), 1.52-1.63 (2H, m),
2.57 (3H, s), 2.67 (2H, t, J=8 Hz), 2.98 (2H, t, J=7 Hz),
3.41 (3H, s), 3.73 (2H, t, J=5 Hz), 3.78 (3H, s),
3.97 (2H, s), 4.11 (2H, t, J=5 Hz), 4.55 (2H, t, J=7 Hz),
6.70-6.79 (2H, m), 6.82 (1H, d, J=8 Hz),
7.18 (2H, d, J=8 Hz), 7.38-7.46 (3H, m),
7.49 (1H, d, J=7 Hz), 7.62 (1H, td, J=8, 1.5 Hz),
7.75 (1H, d, J=8 Hz).

Process 2:

3-{4′-{{4-butyl-6-[3-methoxy-4-(2-methoxyethoxy)phenethoxy]-2-methylpyrimidin-5-yl}methyl}-[1,1′-biphenyl]-2-yl}-1,2,4-oxadiazol-5(4H)-one was obtained as a colorless oil according to the same reaction and treatment as the Processes 2 and 3 of the Example 1 by using 4′-{{4-butyl-6-[3-methoxy-4-(2-methoxyethoxy)phenethoxy]-2-methylpyrimidin-5-yl}methyl}-[1,1′-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.80 (3H, t, J=7 Hz), 1.21-1.29 (2H, m), 1.35-1.45 (2H, m),
2.51 (2H, t, J=7 Hz), 2.55 (3H, s), 3.03 (2H, t, J=6 Hz),
3.20 (3H, s), 3.28 (2H, t, J=5 Hz), 3.64 (3H, s),
3.81 (2H, t, J=5 Hz), 3.89 (2H, s), 4.54 (2H, t, J=6 Hz),
6.66 (2H, d, J=8 Hz), 6.69-6.79 (3H, m),
6.94 (2H, d, J=8 Hz), 7.21-7.33 (1H, m),
7.47 (1H, t, J=8 Hz), 7.57 (1H, t, J=8 Hz),
7.77 (1H, d, J=8 Hz).

### Example 37

Production of 3-{4′-{[4-(2-methoxyethoxy)-2-methyl-6-pentylpyrimidin-5-yl]methyl}-[1,1′-biphenyl]-2-yl}-1,2,4-oxadiazol-5(4H)-one

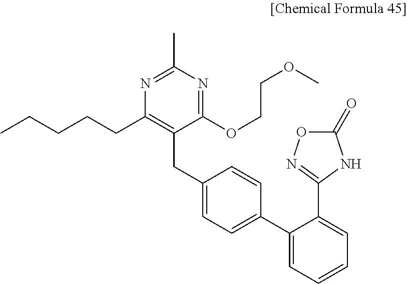

Process 1:

4′-{[4-(2-methoxyethoxy)-2-methyl-6-pentylpyrimidin-5-yl]methyl}-[1,1-biphenyl]-2-carbonitrile was obtained as a colorless oil according to the same reaction and treatment as the Process 1 of the Example 33 according to the same reaction and treatment as the Process 1 of the Example 33 by using 4′-[(2-methyl-6-oxo-4-pentyl-1,6-dihydropyrimidin-5-yl)methyl]-[1,1′-biphenyl]-2-carbonitrile instead of the 4′-[(4-butyl-2-methyl-6-oxo-1,6-dihydropyrimidin-5-yl)methyl]-[1,1′-biphenyl]-2-carbonitrile.

¹H-NMR (CDCl₃) δ:
0.85 (3H, t, J=7 Hz), 1.22-1.38 (4H, m), 1.52-1.63 (2H, m),
2.57 (3H, s), 2.70 (2H, t, J=8 Hz), 3.37 (3H, s),
3.68 (2H, t, J=5 Hz), 4.03 (2H, s), 4.53 (2H, t, J=6 Hz),
7.28 (2H, d, J=8 Hz), 7.37-7.54 (4H, m),
7.62 (1H, td, J=8, 1.5 Hz), 7.74 (1H, d, J=8 Hz).

Process 2:

3-{4′-{[4-(2-methoxyethoxy)-2-methyl-6-pentylpyrimidin-5-yl]methyl}-[1,1′-biphenyl]-2-yl}-1,2,4-oxadiazol-5(4H)-one was obtained as a pale yellow amorphous according to the same reaction and treatment as the Processes 2 and 3 of the Example 1 by using 4′-{[4- (2-methoxyethoxy)-2-methyl-6-pentylpyrimidin-5-yl]methyl}-[1,1-biphenyl]-2-carbonitrile instead of the ethyl 2-{{6-butyl-5-[(2′-cyano-[1,1′-biphenyl]-4-yl)methyl]-2-methylpyrimidin-4-yl}oxy}acetate.

¹H-NMR (CDCl₃) δ:
0.87 (3H, t, J=7 Hz), 1.24-1.38 (4H, m), 1.56-1.65 (2H, m),
2.46 (3H, s), 2.64 (2H, t, J=7 Hz), 3.20 (3H, s),
3.62 (2H, t, J=5 Hz), 3.97 (2H, s), 4.44 (2H, t, J=5 Hz),
7.18 (2H, d, J=8 Hz), 7.21 (2H, d, J=8 Hz),
7.41-7.52 (2H, m), 7.61 (1H, td, J=8, 1.5 Hz),
7.82 (1H, d, J=8 Hz).

### Test Example 1

### Angiotensin II Antagonistic Activity in Isolated Rabbit Blood Vessels

By using a specimen of isolated rabbit blood vessels, antagonistic activity of the compounds of the invention against angiotensin II type 1 receptor was estimated from a dose-response curve of angiotensin II-induced blood vessel contraction. Specifically, the specimen of thoracic aorta ring of a rabbit (New Zealand White: male, 2.4 to 3.0 kg) was suspended in a magnus bath filled with Krebs-Henseleite buffer (composition: 118 mM NaCl, 4.7 mM KCl, 2.55 mM CaCl₂, 1.18 mM MgSO₄, 1.18 mM KH₂PO₄, 24.88 mM NaHCO₃, and 11.1 mM D-glucose), and angiotensin II (10 nM)-induced contraction was obtained in the presence of the compounds of each example (1 nmol/L to 10 μmol/L). During the measurement, the inside temperature of the magnus bath was maintained at 37° C. and the bath was continuously ventilated with a sufficient amount of mixed gas (95% O₂ and 5% CO₂). The angiotensin II-induced contraction was converted into a relative value (%) that is based on the angiotensin II (10 nM)—induced contraction in the absence of the compounds of each example. From the concentration-response curve obtained therefrom, 50% inhibition concentration (IC₅₀ value) was calculated by using SAS Preclinical Package Ver 5.0 (trade name, manufactured by SAS institute Japan Co., Tokyo, Japan), which is a statistical analysis program.

As a result, it was confirmed that the compounds described in the Examples have an angiotensin II antagonistic activity at the concentration of 10 μM or less. The results obtained from the compounds showing preferable results are given in the Table 1. As shown in the Table 1, it was confirmed that the compounds of the invention have a potent angiotensin II antagonistic activity. Further, under the same condition, the angiotensin II inhibition activity of telmisartan as expressed by IC₅₀ value was 0.025 μM.

TABLE 1

| Example No. | IC₅₀ (μM) |
|---|---|
| 1 | 0.81 |
| 2 | 0.23 |
| 4 | 0.29 |
| 5 | 0.06 |
| 6 | 0.13 |
| 15 | 0.77 |
| 22 | 0.10 |
| 26 | 0.38 |
| 28 | 0.64 |
| 30 | 0.23 |
| 33 | 0.48 |

### Test Example 2

### PPARγ Activation Activity

The agonistic activity of the compounds of the invention on PPARγ was measured based on the transfection assay using COST cells (DS Pharma Biomedical Co., Ltd., Osaka, Japan), which are the cell line derived from the kidney of the African green monkey. COST cells were cultured under 5% CO₂ concentration, and DMEM medium containing 10% fetal bovine serum, glutamic acid, and antibiotics was used as a medium.

As an expression vector, a chimera in which DNA binding domain of Ga14, which is a yeast transcription factor, and ligand binding domain of human PPARγ2 are fused, i.e., a fused product between the amino acids 1 to 147 of Gal4 transcription factor and the amino acids 182 to 505 of human PPARγ2, was used. Furthermore, as a reporter vector, a firefly luciferase containing five copies of Gal4 recognition sequence in the promoter region was used. Plasmid transfection to the cells was performed according to a method which uses jetPEI (trade name, manufactured by Funakoshi Co., Ltd., Tokyo, Japan). Furthermore, β-galactosidase expression vector was employed as an internal standard.

After the transfection of the cells, the medium was replaced with a DMEM medium (containing 1% serum) added with the test compound, and the cells were further cultured for 16 hours. After that, the luciferase activity and β-galactosidase activity in the cell lysis solution were measured.

For the present test, dimethylsulfoxide (DMSO) was used for dissolution and dilution of the test compounds, and during the cell treatment, the DMSO concentration in DMEM medium (containing 1% serum) was adjusted to 0.1%. As a positive compound, rosiglitazone (trade name, manufactured by ALEXIS Corporation, Switzerland) was used. The percentage (%) of the luciferase activity of the each test compound (1 to 30 μmol/L) was calculated when the luciferase activity of rosiglitazone (3 to 10 μmol/L) is 100% and the luciferase activity in the absence of the test compound is 0%. The 50% effective concentration of the test compound (EC₅₀, 50% effect concentration) was calculated by using SAS Preclinical Package Ver 5.0 (trade name, manufactured by SAS institute Japan Co., Tokyo, Japan), which is a statistical analysis program.

As a result, it was confirmed that the compounds described in the Examples have a PPARγ activation activity at the concentration of 30 μM or less. The EC₅₀ results are given in the Table 2. As shown in the Table 2, it was confirmed that the compounds of the invention have a potent PPARγ activation activity. Under the same condition, the PPARγ activation activity of telmisartan as expressed by EC₅₀ was 1 μM to 5 μM.

TABLE 2

| Example No. | EC₅₀ (μM) |
|---|---|
| 1 | 1.05 |
| 3 | 2.87 |
| 6 | 0.87 |
| 9 | 2.80 |
| 10 | 1.79 |
| 11 | 0.87 |
| 12 | 0.57 |
| 13 | 1.60 |
| 14 | 0.29 |
| 15 | 1.32 |
| 16 | 0.64 |
| 17 | 0.41 |
| 19 | 0.88 |
| 20 | 0.31 |
| 21 | 0.40 |
| 22 | 0.95 |
| 23 | 0.71 |
| 24 | 1.38 |
| 25 | 0.73 |
| 26 | 2.92 |
| 29 | 2.08 |
| 30 | 1.21 |
| 32 | 0.38 |
| 33 | 0.92 |
| 34 | 1.36 |
| 35 | 1.53 |
| 36 | 0.63 |
| 37 | 0.84 |

From the results obtained above, it was confirmed that the compounds represented by the general formula (I) of the present invention have both a potent angiotensin II receptor antagonistic activity and a PPARγ activation activity. It was confirmed that the compounds have a particularly potent PPARγ activation activity. Thus, it was found that the compounds represented by the formula (I) of the present invention and pharmaceutically acceptable salts thereof are useful as an effective component of a prophylactic and/or therapeutic agent for disorders involved with angiotensin II and PPARγ, for example, hypertension, heart diseases, angina pectoris, cerebrovascular disorders, cerebral circulatory disorders, ischemic peripheral circulatory disorders, renal diseases, arteriosclerosis, inflammatory diseases, type 2 diabetes, diabetic complications, insulin resistance syndrome, syndrome X, metabolic syndrome, and hyperinsulinemia.

1. A compound represented by the formula (I) below, or a salt thereof or a solvate thereof: 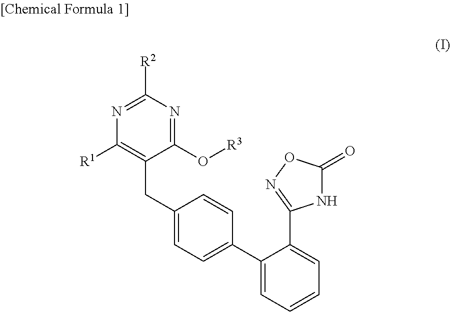
[in the formula, R¹ and R², which may be the same or different from each other, represent a C₁₋₆ alkyl group, and
R³ represents a C₁₋₆ alkyl group which may have one or more substituents selected from the following Group A or a C₃₋₈ cycloalkyl group which may have one or more substituents selected from the following Group B],
Group A: a C₂₋₇ alkoxycarbonyl group; a C₁₋₆ alkoxy group which may have a substituent; a C₁₋₆ alkylthio group; a C₁₋₆ alkylsulfonyl group; a carboxy group; a carbamoyl group which may have one or more substituents; a hydroxy group; an oxo group; a dioxolanyl group; a pyrrolidinyl carbonyl group; a piperidinyl carbonyl group; a morpholinyl carbonyl group; an oxazolyl group which may have one or more substituents; and a C₆₋₁₀ aryl group which may have one or more substituents.
Group B: C₁₋₆ alkyl group; hydroxy group; and an oxo group.

2. The compound according to claim 1, or a salt thereof or a solvate thereof, wherein the C₁₋₆ alkoxy group which may have a substituent is a C₁₋₆ alkoxy group, a C₁₋₆ alkoxy-C₁₋₆ alkoxy group, or a C₆₋₁₀ aryl-C₁₋₆ alkoxy group.

3. The compound according to claim 1, or a salt thereof or a solvate thereof, wherein the carbamoyl group which may have a substituent is a carbamoyl group or a C₁₋₆ alkyl-carbamoyl group.

4. The compound according to claim 1, or a salt thereof or a solvate thereof, wherein the oxazolyl group which may have a substituent is an oxazolyl group, a C₁₋₆ alkyl-oxazolyl group, or a C₆₋₁₀ aryl-oxazolyl group which may be substituted with a C₁₋₆ alkyl group.

5. The compound according to claim 1, or a salt thereof or a solvate thereof, wherein the C₆₋₁₀ aryl group which may have a substituent is a C₆₋₁₀ aryl group or a C₁₋₆ alkyl-C₆₋₁₀ aryl group which may be substituted with a C₁₋₆ alkyl group.

6. The compound according to claim 1, or a salt thereof or a solvate thereof, wherein the compound represented by the formula (I) is a compound selected from the group consisting of
ethyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetate,
2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetic acid,
2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}acetamide,
2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylacetamide,
2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylacetamide,
ethyl 4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoate,
4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}butanoic acid,
4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-butanamide,
4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N-ethylbutanamide,
4-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-N,N-diethylbutanamide,
3-{4′-{[4-butyl-6-(2-ethoxyethoxy)-2-methylpyrimidin-5-yl]methyl}-[1,1′-biphenyl]-2-yl}-1,2,4-oxadiazol-5 (4H)-one,
3-{4′-{[4-butyl-2-methyl-6-(2-propoxyethoxy)pyrimidin-5-yl]methyl}-[1,1′-biphenyl]-2-yl}-1,2,4-oxadiazol-5(4H)-one,
3-{4′-{{4-butyl-6-[2-(2-methoxyethoxy)ethoxy]-2-methylpyrimidin-5-yl} methyl}-[1,1′-biphenyl]-2-yl}-1,2,4-oxadiazol-5(4H)-one,
3-{4′-{{4-[2-(benzyloxy)ethoxy]-6-butyl-2-methylpyrimidin-5-yl}methyl}-[1,1′-biphenyl]-2-yl}-1,2,4-oxadiazol-5(4H)-one,
3-{4′-{[4-butyl-6-(2-hydroxyethoxy)-2-methylpyrimidin-5-yl]methyl}-[1,1′-biphenyl]-2-yl}-1,2,4-oxadiazol-5 (4H)-one,
3-{4′-{[4-butyl-6-(2-isopropoxyethoxy)-2-methylpyrimidin-5-yl]methyl}-[1,1′-biphenyl]-2-yl}-1,2,4-oxadiazol-5(4H)-one,
3-{4′-{{4-butyl-2-methyl-6[2-(methylthio)ethoxy]pyrimidin-5-yl}methyl}-[1,1′-biphenyl]-2-yl}-1,2,4-oxadiazol-5(4H)-one,
3-{4′-{{4-butyl-2-methyl-6-[2-(methylsulfonyl}ethoxy]pyrimidin-5-yl}methyl}-[1,1′-biphenyl]-2-yl}-1,2,4-oxadiazol-5(4H)-one,
3-{4′-{[4-butyl-6-(2,2-dimethoxyethoxy)-2-methylpyrimidin-5-yl]methyl}-[1,1′-biphenyl]-2-yl}-1,2,4-oxadiazol-5(4H)-one,
3-{4′-{[4-butyl-6-(2,2-diethoxyethoxy)-2-methylpyrimidin-5-yl]methyl}-[1,1′-biphenyl]-2-yl}-1,2,4-oxadiazol-5(4H)-one,
3-{4′-{[4-butyl-6-(2-hydroxypropoxy)-2-methylpyrimidin-5-yl]methyl}-[1,1′-biphenyl]-2-yl}-1,2,4-oxadiazol-5(4H)-one,
3-{4′-{[4-butyl-2-methyl-6-(2-oxopropoxy)pyrimidin-5-yl]methyl}-[1,1′-biphenyl]-2-yl}-1,2,4-oxadiazol-5 (4H)-one,
3-{4′-{[4-butyl-6-(4-methoxybutoxy)-2-methylpyrimidin-5-yl]methyl}-[1,1′-biphenyl]-2-yl}-1,2,4-oxadiazol-5 (4H)-one,
3-{4′-{{4-[(1,3-dioxolan-2-yl)methoxy]-6-butyl-2-methylpyrimidin-5-yl}methyl}-[1,1′-biphenyl]-2-yl}-1,2,4-oxadiazol-5(4H)-one,
methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-2-phenylacetate,
2-{{6-butyl-2-methyl-5-{[2′-(5-oxo-4,5-dihydro-1,2,4-oxadiazol-3-yl)-[1,1-biphenyl]-4-yl]methyl}pyrimidin-4-yl}oxy}-2-phenylacetic acid,
methyl 2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionate,
2-{{6-butyl-2-methyl-5-{[2′-(5-oxo-4,5-dihydro-1,2,4-oxadiazol-3-yl)-[1,1′-biphenyl]-4-yl]methyl}pyrimidin-4-yl}oxy}-3-phenylpropionic acid,
3-{4′-{{4-butyl-2-methyl-6-[2-oxo-2-(pyrrolidin-1-yl)ethoxy]pyrimidin-5-yl}methyl}-[1,1′-biphenyl]-2-yl}-1,2,4-oxadiazol-5(4H)-one,
3-{4′-{{4-butyl-2-methyl-6-[2-oxo-2-(piperidin-1-yl)ethoxy]pyrimidin-5-yl}methyl}-[1,1′-biphenyl]-2-yl}-1,2,4-oxadiazol-5(4H)-one,
3-{4′-{[4-butyl-2-methyl-6-(2-morpholino-2-oxoethoxy)pyrimidin-5-yl]methyl}-[1,1′-biphenyl]-2-yl}-1,2,4-oxadiazol-5(4H)-one,
3-{4′-{{4-butyl-2-methyl-6-{[5-methyl-2-(p-tolypoxazol-4-yl]methoxy}pyrimidin-5-yl}methyl}-[1,1′-biphenyl]-2-yl}-1,2,4-oxadiazol-5(4H)-one,
3-{4′-{[4-butyl-6-(2-methoxyethoxy)-2-methylpyrimidin-5-yl]methyl}-[1,1′-biphenyl]-2-yl}-1,2,4-oxadiazol-5 (4H)-one,
3-{4′-{{4-butyl-6-[(4-hydroxycyclohexyl)oxy]-2-methylpyrimidin-5-yl]methyl}-[1,1′-biphenyl]-2-yl}-1,2,4-oxadiazol-5(4H)-one,
3-{4′-{{4-butyl-2-methyl-6-[(4-oxocyclohexyl)oxy]pyrimidin-5}-yl}methyl}-[1,1′-biphenyl]-2-yl}-1,2,4-oxadiazol-5(4H)-one,
3-{4′-{{4-butyl-6-[3-methoxy-4-(2-methoxyethoxy)phenethoxy]-2-methylpyrimidin-5-yl}methyl}-[1,1′-biphenyl]-2-yl}-1,2,4-oxadiazol-5(4H)-one, and
3-{4′-{[4-(2-methoxyethoxy)-2-methyl-6-pentylpyrimidin-5-yl]methyl}-[1,1′-biphenyl]-2-yl}-1,2,4-oxadiazol-5(4H)-one.

7. A pharmaceutical composition comprising the compound described in claim 1, or a salt thereof, or a solvate thereof, and a pharmaceutically acceptable carrier.

8. A pharmaceutical composition having both angiotensin II receptor antagonistic activity and PPARγ activation activity in which the compound described in claim 1, or a salt thereof, or a solvate thereof is comprised as an effective component.

9. A prophylactic and/or therapeutic agent for a circulatory disorder which comprises as an effective component the compound described in claim 1, or a salt thereof, or a solvate thereof.

10. The agent according to claim 9, in which the circulatory disorder is hypertension, heart diseases, angina pectoris, cerebrovascular disorders, cerebral circulatory disorders, ischemic peripheral circulatory disorders, renal diseases, or arteriosclerosis.

11. A prophylactic and/or therapeutic agent for a metabolic disorder which comprises as an effective component the compound described in claim 1, or a salt thereof, or a solvate thereof.

12. The agent according to claim 11, in which the metabolic disorder is type 2 diabetes, diabetic retinopathy, diabetic neuropathy, diabetic nephropathy, insulin resistance syndrome, metabolic syndrome, or hyperinsulinemia.

13. A method of preventing and/or treating a circulatory disorder, the method comprising administering an effective amount of the compound described in claim 1, or a salt thereof, or a solvate thereof to a patient who is in need of treatment.

14. A method of preventing and/or treating a metabolic disorder, the method comprising administering an effective amount of the compound described in claim 1, or a salt thereof, or a solvate thereof to a patient who is in need of treatment.

15. Use of the compound described in claim 1, or a salt thereof, or a solvate thereof for producing a preparation for preventing and/or treating a circulatory disorder.

16. Use of the compound described in claim 1, or a salt thereof, or a solvate thereof for producing a preparation for preventing and/or treating a metabolic disorder.

17. The compound described in claim 1, or a salt thereof, or a solvate thereof as an agent for prevention and/or treatment having both angiotensin II receptor antagonistic activity and PPARγ activation activity.


SEQUENCE LISTING (8 sequences)

SEQ ID NO: 1 - DNA, 33 bp, Artificial Sequence
  primer (PAG-U)
gtgggtaccgaaatgaccatggttgacacagag

SEQ ID NO: 2 - DNA, 33 bp, Artificial Sequence
  primer (PAG-L)
ggggtcgaccaggactctctgctagtacaagtc

SEQ ID NO: 3 - DNA, 33 bp, Artificial Sequence
  primer (XRA-U)
ttagaattcgacatggacaccaaacatttcctg

SEQ ID NO: 4 - DNA, 33 bp, Artificial Sequence
  primer (XRA-L)
cccctcgagctaagtcatttggtgcggcgcctc

SEQ ID NO: 5 - DNA, 36 bp, Artificial Sequence
  5′-phosphorylated DNA (PPRE-U)
tcgacaggggaccaggacaaaggtcacgttcgggag

SEQ ID NO: 6 - DNA, 36 bp, Artificial Sequence
  5′-phosphorylated DNA (PPRE-L)
tcgactcccgaacgtgacctttgtcctggtcccctg

SEQ ID NO: 7 - DNA, 28 bp, Artificial Sequence
  primer (TK-U)
cccagatctccccagcgtcttgtcattg

SEQ ID NO: 8 - DNA, 28 bp, Artificial Sequence
  primer (TK-L)
tcaccatggtcaagcttttaagcgggtc